(19) United States
(12) Patent Application Publication
(10) Pub. No.: US 2010/0056445 A1
(43) Pub. Date: Mar. 4, 2010

(54) ADIPONECTIN FOR THE TREATMENT AND DIAGNOSIS OF ALBUMINURIA

(75) Inventors: Kumar Sharma, Del Mar, CA (US); Barry Goldstein, Bala Cynwyd, PA (US)

Correspondence Address: DAVID S. RESNICK, NIXON PEABODY LLP, 100 SUMMER STREET, BOSTON, MA, 02110-2131, US

(73) Assignee: THOMAS JEFFERSON UNIVERSITY, Philadelphia, PA (US)

(21) Appl. No.: 12/446,773

(22) PCT Filed: Nov. 7, 2007
(86) PCT No.: PCT/US2007/023466

§ 371 Date: Apr. 23, 2009

Related U.S. Application Data
(60) Provisional application No. 60/857,459, filed on Nov. 7, 2006.

(51) Int. Cl. A61K 38/17; C12Q 1/68; G01N 33/53; A61K 31/7088; G01N 33/00

(52) U.S. Cl. 514/12; 435/6; 435/7.1; 514/44 R; 436/86; 436/94

(57) ABSTRACT

Disclosed are methods relating to the treatment and/or prevention of kidney disorders, especially kidney disorders characterized by or involving albuminuria. Methods described include the administration of an adiponectin polypeptide or a nucleic acid encoding such a polypeptide to treat or prevent the development of albuminuria. Also described are methods in which adiponectin is measured as a predictor of a subject's likelihood of having or developing a kidney disorder characterized by or involving albuminuria. Also described are methods of treating or preventing a kidney disorder involving administering an AMPK agonist and/or an inhibitor of Nox4 activity.

### CROSS REFERENCED APPLICATIONS

This application claims the benefit under 35 U.S.C. 119(e) of U.S. Provisional Application Ser. No. 60/857,459 filed on Nov. 7, 2006 the contents of which are incorporated herein by reference.

### GOVERNMENT SUPPORT

This application was made with Government support under Grant No. HL-51586 awarded by the National Institutes for Health (NIH). The Government of the United States has certain rights in the invention.

### FIELD

The invention relates to the use of adiponectin as a diagnostic and as a protective agent for albuminuria.

### BACKGROUND

Chronic kidney disease and albuminuria have recently been recognized to be among the most significant clinical risk factors for cardiovascular disease (CVD), hospitalization, and all-cause mortality (1-5). Microalbuminuria, resulting from leakage of albumin across the glomerular podocyte filtration barrier into the urine, is considered a clinical window for a more generalized dysfunction in the systemic vasculature indicating a heightened risk of cardiovascular disease (CVD) (1, 2, 6). Renal dysfunction may contribute to overall CVD by also promoting vascular thickening and vascular calcification (7); as well as by activating inflammatory pathways (8). It has also been recognized that insulin resistance is closely associated with early decline in renal function and albuminuria (1, 9). Recently, albuminuria in the so-called high normal range (10-30 ug/mg) has been identified as a risk factor for cardiovascular disease (44). Renal dysfunction may contribute to overall CVD by also promoting vascular thickening and vascular calcification (7), as well as by activating inflammatory pathways (8). It has also been recognized that insulin resistance is closely associated with oxidant stress, early decline in renal function, and albuminuria (1, 9). Despite the close relationships demonstrated to exist between cardiovascular disease and kidney dysfunction the mechanism linking these entities together has not been elucidated.

Adiponectin is a recently identified circulating plasma protein that has been recognized to be a key predictive factor for cardiovascular mortality in patients with renal dysfunction (8, 10). Adiponectin, a 30 kDa protein primarily secreted by adipocytes, has largely beneficial effects as it improves insulin sensitivity and decreases the adverse effects of inflammatory mediators in vascular cells (11, 12). Adiponectin, also referred to as ACRP30, AdipoQ and gelatin-binding protein-28 (41-43), is an adipocyte-specific cytokine. Plasma adiponectin levels are reduced with increasing visceral obesity and are tightly correlated with insulin resistance and the development of type 2 diabetes mellitus (13). In patients with type 1 diabetes low plasma adiponectin levels were predictive of the development of coronary artery calcification (14, 15) suggesting an important role for adiponectin in development of macrovascular disease. Adiponectin may also be cleaved into a collagenous and globular domain. The globular form of adiponectin may be derived from cleavage by neutrophil elastase and has been found in both human and mouse plasma (45).

African Americans have a disproportionate and excessive representation of ESRD (33). African Americans also have high rates of obesity which heightens risk for kidney and cardiovascular disease. Low adiponectin levels have been identified in obese African Americans and are also associated with susceptibility to diabetes (16, 17). In the African American (AA) population; low plasma adiponectin levels have been reported in obese subjects and may be predictive of the development of type 2 diabetes (16, 17). Although both adiponectin levels and albuminuria are associated with CVD and kidney dysfunction, studies linking adiponectin with albuminuria have been inconclusive (20-22). Furthermore, a role for adiponectin in relation to albuminuria has not been established.

### SUMMARY

The inventors of the present invention have surprisingly discovered that adiponectin is inversely related to the degree of albuminuria in obese African Americans without diabetes or overt kidney disease. One aspect of the invention provides for methods to detect levels of adiponectin in a biological sample from a subject to determine the likelihood of a subject having or at risk of developing albuminuria.

Importantly, the inventors have also discovered that adiponectin plays a protective role to reduce albuminuria by directly affecting podocyte function via the AMPK pathway. More specifically, the inventors provide methods and compositions for the treatment of subjects with or at risk of developing albuminuria. In particular, the compositions and methods comprise the administration of adiponectin, adiponectin agonists or agonists of the 5′-AMP activated protein kinase (AMPK)-dependent pathway for the treatment of albuminuria.

In one embodiment, methods to detect adiponectin in a biological sample are disclosed. Methods of the present invention comprise detecting adiponectin levels, which, if they fall below a baseline level, indicates that the subject is at risk of developing or having albuminuria. The methods of the invention provide means to detect adiponectin in a biological sample, typically obtained from the subject, for example but not limited to a blood, plasma or serum sample.

In one embodiment, the methods of the invention involve the detection of levels of adiponectin polypeptide or protein, or fragments of adiponectin protein. In such an embodiment, the methods for example include but are not limited to; electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunohistochemistry, immunocytochemistry, FACS scanning, immunoblotting, immunoprecipitation, affinity chromatography, immunoelectrophoresis, radioimmunoassay (RIA), enzyme-linked immunosorbent assays (ELISAs), immunofluorescent assays, Western blotting, and the like.

In another embodiment, the methods of the invention are methods to detect levels of adiponectin nucleic acid, for example adiponectin gene transcription, for example RNA. In such an embodiment, the methods for example include but are not limited to amplification-based assays; PCR-based methods; microarray and MassArray based systems; hybridization based methods; northern blots; and fluorescence based in situ hybridization systems.

In another embodiment, the invention provides methods and compositions to treat a subject having or at risk of developing albuminuria. In one such embodiment, the method provides administration of an effective amount of a pharmaceutical composition comprising an adiponectin polypeptide, a variant of adiponectin, or active fragments thereof to a subject. In another embodiment, the invention provides a pharmaceutical composition comprising an agonist of adiponectin. In another embodiment, the method provides a pharmaceutical composition comprising an AMPK polypeptide, an AMPK agonist or analogue of AMPK or active fragment thereof, or activators of the AMPK pathway.

In another embodiment, the invention provides methods to treat albuminuria by administering a pharmaceutical composition comprising a nucleic acid encoding adiponectin, a variant of adiponectin, or a an active portion of adiponectin. In a related embodiment, the invention provides methods for producing a recombinant DNA molecule comprising a nucleic acid encoding adiponectin, a variant of adiponectin, or an active portion of adiponectin.

The invention also provides methods for production of a pharmaceutical composition for the treatment of albuminuria, and methods for administration of the pharmaceutical composition to a subject at risk of developing or having albuminuria.

In another embodiment, the methods of the invention also encompass methods to screen for adiponectin and/or AMPK agonists or activators of the AMPK pathway.

In further embodiment, the methods of the invention also provide for kits to screen subjects for levels of adiponectin.

In one aspect, then, disclosed herein is a method for decreasing the risk of developing, or reducing the effects of a kidney disorder in a subject, the method comprising administering to the subject an effective amount of a pharmaceutical composition comprising an adiponectin polypeptide and a pharmaceutically acceptable carrier. In one embodiment of this and other aspects of the methods disclosed herein, the method includes the step, prior to administering the composition, of testing a urine sample from the subject; proteinuria or albuminuria in the subject would be an indication for administering the composition. Similarly, in another embodiment, the method can comprise measuring a level of an adiponectin polypeptide or RNA encoding adiponectin in a sample from the subject; a reduced level of adiponectin polypeptide or RNA in such sample, relative to a control or reference level of adiponectin in a healthy individual would also be an indication for administering the composition, as a way to treat or prevent albuminuria. In another embodiment, the subject is tested for both adiponectin and albuminuria prior to the commencement of treatment.

In one embodiment, the kidney disorder is albuminuria.

In another embodiment, administration is prior to the onset of the kidney disorder.

In another embodiment, administration is post onset of the kidney disorder.

In another embodiment, administration is substantially concurrent with the kidney disorder.

In another embodiment, administration is within 24 hours after the onset of the kidney disorder.

In another embodiment, the adiponectin polypeptide is a trimer. Alternatively, or in addition, the adiponectin polypeptide can be the globular domain of adiponectin. In another embodiment, the adiponectin polypeptide comprises a human adiponectin polypeptide. In another embodiment, the globular domain comprises, or alternatively, consists of a polypeptide having the sequence of SEQ ID NO: 2. In another embodiment, the adiponectin polypeptide comprises, or alternatively, consists of a polypeptide having the sequence of SEQ ID NO: 1.

In another embodiment, the subject is suffering from a condition selected from the group consisting of: hypertension; obesity; and kidney disease.

In another aspect, described herein is a method for decreasing the risk of developing, or reducing the effects of a kidney disorder in a subject, the method comprising administering to the subject an effective amount of a pharmaceutical composition comprising a nucleic acid construct comprising sequence encoding an adiponectin polypeptide operatively linked to control sequences sufficient for the expression of the adiponectin polypeptide.

In one embodiment of this and other aspects of the methods disclosed herein, the method includes the step, prior to administering the composition, of testing a urine sample from the subject; proteinuria or albuminuria in the subject would be an indication for administering the composition. Similarly, in another embodiment, the method can comprise measuring a level of an adiponectin polypeptide or RNA encoding adiponectin in a sample from the subject; a reduced level of adiponectin polypeptide or RNA in such sample, relative to a control or reference level of adiponectin in a healthy individual would also be an indication for administering the composition, as a way to treat or prevent albuminuria. In another embodiment, the subject is tested for both adiponectin and albuminuria prior to the commencement of treatment.

In one embodiment, the kidney disorder is albuminuria.

In another embodiment, the control sequences are inducible.

In another embodiment, the nucleic acid construct is comprised by a viral vector.

In another embodiment, administration is prior to the onset of the kidney disorder. In another embodiment, administration is post onset of the kidney disorder. In another embodiment, administration is substantially concurrent with the kidney disorder. In another embodiment, administration is within 24 hours after the onset of the kidney disorder.

In one embodiment, the adiponectin polypeptide can be the globular domain of adiponectin. In another embodiment, the adiponectin polypeptide comprises a human adiponectin polypeptide. In another embodiment, the globular domain comprises, or alternatively, consists of a polypeptide having the sequence of SEQ ID NO: 2. In another embodiment, the adiponectin polypeptide comprises, or alternatively, consists of a polypeptide having the sequence of SEQ ID NO: 1. In another embodiment, the nucleic acid construct comprises polypeptide coding sequence encoded in SEQ ID NO: 3.

In another embodiment, the subject is suffering from a condition selected from the group consisting of: hypertension; obesity; and kidney disease.

In another aspect, disclosed herein is a method for treating albuminuria, the method comprising administering to a subject in need thereof a pharmaceutical composition comprising an adiponectin polypeptide, in a pharmaceutically acceptable carrier.

In another aspect, disclosed herein is a method for treating albuminuria, the method comprising administering to a subject in need thereof a pharmaceutical composition comprising a nucleic acid encoding an adiponectin polypeptide, operatively liked to control sequences sufficient for the expression of said adiponectin polypeptide, said nucleic acid in a pharmaceutically acceptable carrier.

In another aspect, a method is disclosed herein for treating albuminuria, the method comprising administering to a subject in need thereof an inhibitor of Nox4 activity.

In another aspect, a method is disclosed herein for treating or reducing the risk of developing a kidney disorder, the method comprising administering to a subject in need thereof an effective amount of a pharmaceutical composition comprising an AMPK agonist in a pharmaceutically acceptable carrier. In one embodiment, the AMPK agonist comprises AICAR.

In another aspect, disclosed herein is a method of identifying a subject having increased likelihood of having or developing a kidney disorder, the method comprising measuring a level of adiponectin or RNA encoding adiponectin in a biological sample obtained from a subject and comparing said level to a baseline level, wherein when the measured level of adiponectin or RNA encoding adiponectin is below said baseline level, the subject is identified as having an increased likelihood of having or being at risk of developing a kidney disorder. In one embodiment, the kidney disorder is albuminuria. In another embodiment, the subject has a disorder selected from the group consisting of: hypertension; obesity; glucose intolerance; and diabetes.

In one embodiment, the method biological sample is selected from the group consisting of serum, whole blood, plasma, urine, and a tissue sample. In another embodiment, the biological sample is a urine sample.

In another embodiment, measuring a level of adiponectin comprises measuring an adiponectin polypeptide. In another embodiment, measuring a level of adiponectin comprises an ELISA. In another embodiment, measuring a level of adiponectin RNA measures a level of messenger RNA (mRNA). The level of mRNA can be measured, for example, by reverse transcription-polymerase chain reaction (RT-PCR).

In another aspect, disclosed herein is a kit for treating a subject suffering from a kidney disorder, the kit comprising a nucleic acid containing a segment encoding an adiponectin polypeptide, operatively linked to control sequences sufficient for the expression of said adiponectin polypeptide, or a pharmaceutical composition comprising an adiponectin polypeptide, and a pharmaceutically acceptable carrier or excipient. In one embodiment, the nucleic acid is comprised by a viral vector.

### BRIEF DESCRIPTION OF THE FIGURES

FIG. 1 shows the association between albuminuria and plasma adiponectin levels in obese African Americans. Regression between adiponectin levels and urine albumin/creatinine ratios. Confidence intervals and Spearman's correlation co-efficient and p values for all variables tested are listed in Table 2.

FIG. 2 shows adiponectin knockout mice exhibit increased albuminuria and podocyte foot process effacement. A) Urine albumin/creatinine ratios in 2 month old adiponectin KO mice (AdKO) are significantly increased as compared to wild type (WI) mice (mean±SEM, ***p<0001 vs. wild type, n=10 per group). B) Podocyte foot processes are segmentally effaced in adiponectin KO (AdKO) mouse kidneys by electron microscopy (image magnification 4000×). Arrows points to areas of foot process effacement. Photograph representative of 10 EM images per kidney from 2 mice per group.

FIG. 3 shows adiponectin inhibits permeability across podocytes via AMPK. 3A) Permeability of albumin across a podocyte monolayer was reduced by adiponectin (adipo) at 3 μg/ml (data presented as % of control value with mean±SEM, **p<001 vs. control, n=3 per group). Cells were treated as described in methods section and permeability assessed by albumin concentration across podocyte monolayer. 3B) AMPK activity was increased by adiponectin treatment in podocytes as demonstrated by immunoblotting and 3C) confocal microscopy. AMPK activity was assessed with an antibody specific for p-Thr172 on AMPKα subunit. Representative blots and confocal photographs from 3 separate experiments. 3D) AMPK inhibitor (ARA-A) blocked protective effect of adiponectin across podocyte monolayer. Cells were treated as described in 3A and methods section. Podocytes pre-treated with ARA-A prior to addition of adiponectin blocked effect of adiponectin and increased permeability (data presented as % control, mean±SEM; p<0.01 vs. control and p<0.001 vs. adipo alone, n=6 per group).

FIG. 4 shows adiponectin administration restores normal albuminuria and increases AMPK activity in podocytes. 4A) Adiponectin KO (AdKO) mice at 4 months of age were treated with recombinant globular adiponectin (gAd) for 10 days and urine albumin/creatinine ratios were measured with and without gAd treatment (mean±SEM, n=10 per group). 4B) Podocyte foot process fusion in adiponectin KO mice were reduced with gAd treatment (image magnification 4000×, compare with FIG. 2B). Photograph representative of 10 EM images per kidney from 2 mice per group. 4C) AMPK activity was reduced in glomerular podocytes of adiponectin KO mice and increased by adiponectin treatment (light microscopy immunostain, 40×). Arrows point to podocytes that are p-AMPKα positive and insets show higher magnification of the same cells. Mouse kidneys were immunostained with antibody specific for p-AMPKα as described in methods section. Photomicrographs are representative of 50 glomeruli from each mouse kidney per group (n=4 per group). 4D) Quantitation of number of p-AMPKα positive cells per glomerulus in each group (mean±SEM, n=4 mice per group).

FIG. 5 shows adiponectin knockout mice exhibit increased albuminuria, oxidant stress and podocyte dysfunction. 5A) Urine albumin/creatinine ratios in adiponectin KO mice (Ad−/−) are significantly increased as compared to corresponding age-matched wild type (WT) mice at 1, 2, 3 and 4 months of age (mean±SEM, *p<0.01 vs. corresponding age-matched wild type, n=10 per group). 5B) Wild type and Ad−/− mice were made diabetic with low dose streptozotocin and urine albumin/creatinine ratios measured before, and 2 and 4 months of diabetes. Albuminuria was significantly increased in Ad−/− mice with diabetes compared to corresponding wild type diabetic groups (mean±SEM, *p<0.05 vs. WT control, **p<0.05 vs WT DM at 2 months of diabetes, ***p<0.05 vs WT DM at 4 months of diabetes, n=5-10 per group). 5C) Urinary hydrogen peroxide/creatinine levels are significantly increased in Ad−/− mice with and without diabetes (mean±SEM, *p<0.05 vs. WT control, **p<0.05 vs WT DM at 2 months of diabetes, n=10 per group). 5D) Podocyte foot processes are segmentally effaced in Ad−/− mouse kidneys by electron microscopy (image magnification 5000×). Arrows points to areas of normal foot processes in wild type kidneys (left panel) and areas of foot process effacement in Ad−/− glomeruli (right panel). Photograph representative of 10 EM images per kidney from 2 mice per group.

FIG. 6 shows adiponectin inhibits permeability across podocyte monolayer. 6A) Permeability of albumin across a podocyte monolayer was reduced by globular adiponectin (gAd) or full length adiponectin (fAd) at 3 μg/ml (data presented as % of control value with mean±SEM, *p<0.01 vs. control, n=3 per group). Cells were treated as described in methods section and permeability assessed by albumin concentration across podocyte monolayer. Expression of AdipoR1 (6B) and AdipoR2 (6C) by real time PCR in wild type mouse liver, kidney and differentiated podocytes. Gene expression depicted in relation to β-actin and expressed as 100% in mouse liver.

FIG. 7 shows AMPK activity is increased by adiponectin and regulates podocyte permeability. Treatment of podocytes with globular adiponectin (3 μg/ml, 24 h) increases AMPK activity as demonstrated by confocal microscopy (7A) and immunoblotting (7B). AMPK activity was assessed with antibodies specific for p-AMPKa subunit. Total AMPKa was measured with antibody for AMPKa as a loading control (B, lower panel). Effect of high glucose (HG) to decrease AMPK activity was blocked by adiponectin. Representative confocal photographs and immunoblots from 3 separate experiments. 7C) Albumin permeability was decreased by the AMPK activator (AICAR, 1 mM) and increased by the AMPK inhibitor (ARA). The effect of adiponectin to reduce permeability was also blocked by ARA. Cells were treated as described in 3A and methods section. (Data presented as % control, mean±SEM; p<0.01 vs. control and p<0.001 vs. gAd alone, n=5 per group.)

FIG. 8 shows adiponectin restores normoalbuminuria and increases AMPK activity. A) Adiponectin KO (Ad−/−) mice at 4 months of age were treated with saline, globular adiponectin (gAd), full length adiponectin (fAd) or AICAR, and urine albumin/creatinine ratios were measured. gAd, fAd and AICAR treatment significantly decreased urine albumin/creatinine to the control values seen in WT mice (*p<0.05 vs WT, #p<0.05 vs Ad−/−, mean±SEM, n=7-10 per group). 8B) Podocyte foot process fusion in Ad−/− mice were reduced with gAd treatment (image magnification 5000×, compare with FIG. 2D). 8C) AMPK activity was demonstrated in normal glomerular podocytes by double labeling with P-AMPK antibody and podocytespecific synaptopodin antibody. 8D, 8E) AMPK activity was reduced in glomerulus of Ad−/− mice and increased by adiponectin treatment. Mouse kidneys (WT left panel, Ad−/− middle panel, and Ad−/− treated with gAd right panel) were immunostained with antibody specific for p-AMPKa as described in methods section (light microscopy immunostain, 40×). Arrows point to podocytes that are p-AMPKa positive and insets show higher magnification of the same cells. Photomicrographs are representative of 50 glomeruli from each mouse kidney per group (n=4 per group). 8E) Quantitation of number of p-AMPKa positive cells per glomerulus in each group (*p<0.05 vs WT, **p<0.05 vs Ad−/−, mean±SEM, n=4 mice per group).

FIG. 9 shows regulation of oxidant stress and Nox4 by adiponectin. 9A) Urinary levels of hydrogen peroxide were reduced by gAd, fAd or AICAR treatment in Ad−/− mice (*p<0.05 vs WT, #p<0.05 vs Ad−/−, mean±SEM, n=7-10 per group). 9B) Kidney Nox4 mRNA levels were increased in Ad−/− kidneys and reduced with gAd treatment. (Data presented as % control, *p<0.05 vs WT, **p<0.05 vs Ad−/−, mean±SEM, n=5 per group). 9C) Nox4 is present in podocytes, as well as other glomerular cells and tubular cells, as demonstrated by double labeling with synaptopodin in WT kidney. 9D) Nox4 protein is increased in glomerular cells of Ad−/− kidneys and reduced with gAd treatment (light microscopy immunostain, 40×). (Photomicrographs are representative of 50 glomeruli from each mouse kidney per group (n=4 per group)).

FIG. 10 shows podocyte Nox4 is increased by high glucose and reduced by adiponectin or AICAR. Panel 10 shows podocytes grown in presence of gAd (3 μg/ml, 24 h) had suppression of Nox4 in presence of normal glucose (NG) or high glucose (HG). Transferred proteins were immunoblotted with antibody to Nox4 (upper panel) and β-actin (lower panel). 10B) Similar studies were performed with podocytes grown on coverslips demonstrating reduction of Nox4 protein. 10C) AMPK activation with AICAR had a similar degree of reduction of Nox4 protein as gAd in podocytes grown in high glucose (HG). Representative immunoblots and confocal photographs from 3 separate experiments.

### DETAILED DESCRIPTION

The present invention is based on the surprising discovery that adiponectin levels are inversely related to microalbuminuria in obese African Americans without diabetes or overt kidney disease. Although not wishing to be bound by theory, the inventors have discovered that adiponectin plays a protective role to reduce albuminuria by directly affecting podocyte function via the AMPK pathway. The inventors discovered that administration of adiponectin to the Adiponectin knockout (AdKO) mice that have podocyte dysfunction and increased levels of albuminuria, increased podocyte AMPK activity, improved podocyte foot processes, and normalized albuminuria. Accordingly, the present invention provides methods to treat or reduce the risk of developing albuminuria by administration of adiponectin or adiponectin agonists, or activation of the AMPK pathway.

Further, since chronic kidney disease and albuminuria are important risk factors in cardiovascular diseases (CVD) and CVD mortality, the identification of low adiponectin levels and increased albuminuria in subjects identifies a population of high risk profile to progressive renal disease as well as associated cardiovascular disease. Accordingly, the present invention provides methods for screening subjects for levels of adiponectin to prognose subjects at risk of, or likely to developing kidney disease and cardiovascular disease. In such embodiments, adiponectin is administered to subjects with low levels of adiponectin, either prior to or concurrent with albuminuria.

### DEFINITIONS

The terms “adiponectin” or “ACRP30” or “Acrp30” or “AdipoQ gelatin-binding protein-28” or “apM1” are used interchangeably herein, and referred to as “adiponectin” herein and throughout the specification. The terms refer to the gene product or nucleic acid sequence encoding adiponectin that is an adipocyte-specific cytokine. For reference purposes only and as an example, and not intended to limit the scope of the invention, the human form of adiponectin has the accession number for the human adiponectin gene transcript NM_{—}004797, and the rat accession number is NM_{—}144744. Protein accession numbers are NP_{—}004788 and NP_{—}653345 for human and rat respectively. See also, U.S. Pat. No. 5,869,330; US20020132773; US200230147855 and US200230176328. Normal adiponectin levels in serum in humans range from about 5 μg/ml to about 30 μg/ml.

The term “adiponectin polypeptide,” as the term is used herein, refers to a polypeptide that comprises at least 15 contiguous amino acids of adiponectin and functions to reduce albuminuria when administered to an adiponectin −/− mouse. Such an adiponectin polypeptide thus encompasses “an active portion” of adiponectin. The term encompasses full length adiponectin polypeptide, e.g., a polypeptide corresponding to SEQ ID NO: 1. The term “adiponectin polypeptide” also encompasses, for example, adiponectin polypeptides that correspond to the globular domain and retain the ability to reduce albuminuria when introduced to adiponectin −/− mice, including, but not limited to those described in U.S. published patent application No. 20060281151.

Full length adiponectin is a 30 kD glycoprotein having an N-terminal collagen-like domain, approximately residues 1-100, containing multiple G-X-X-G repeats, and a C-terminal domain, approximately residues 108-244, structurally resembling the globular portions of the C1Q and TNF superfamily members. At least two proteolytic cleavage sites are located between the collagen and C1Q-like domains. Both full length and proteolytically cleaved forms are found in human serum. Globular head domain cleavage fragments of adiponectin form trimeric structures, while full length adiponectin is capable of forming trimers, hexamers, and additional higher order oligomers. Mutation of the cysteine residue located in the collagen domain (conserved in all known mammalian adiponectin) abolishes hexamer and high-order oligomer formation.

Homologous proteins to adiponectin include, but are not limited to, mouse Clq/TNF-.alpha. Related Proteins 1 (CTRP1), CTRP2, CTRP3, CTRP4, CTRP5, CTRP6 and CTRP7. At least one of these proteins (CTRP2) is able to stimulate fatty acid oxidation in skeletal muscle, thus resembling the functional properties of adiponectin (Wong et al. (2004) Proc. Natl. Acad. Sci. 101:10302-7, entirely incorporated by reference).

Several adiponectin polymorphisms have been discovered within particular human populations. The phenotype depends on the position of the mutation. For example, the G84R, G90S, Y111H, and I164T mutations cause diabetes and hypoadiponectinemia as a result of a failure to form higher order oligomers that are likely important in regulating insulin sensitivity by the liver (Waki et al. (2003) J. Biol. Chem. 278:40352-63, entirely incorporated by reference). Additional polymorphisms include R221S and H241P.

The term “globular domain,” when used herein in the context of adiponectin, refers to the portion of the protein that comprises the globular component of the adiponectin protein. As noted above, the “globular domain” corresponds to approximately amino acids 108-244 of the full length wild-type human adiponectin polypeptide. In one embodiment, the globular domain consists of amino acids 111-242 of the full length wild-type human adiponectin polypeptide at GenBank Accession No. NP_{—}004788 (SEQ ID NO: 1).

The term “albuminuria” used herein refers to the presence of protein in the urine, principally albumin. This often leads to or is indicative of a disease, but is not necessary limited to incidence where albuminuria produces a disease. Albuminuria is meant to encompass all forms of albuminuria, including but not limited to physiologic albuminuria; functional albuminuria; and albuminuria of athletes, which relates to a form of functional albuminuria following excessive muscular exertion. Further, albuminuria covers benign albuminuria (also known as essential albuminuria), which refers to types or albuminuria that are not the result of pathologic changes in the kidneys. Albuminuria also covers pathologic albuminuria, for example levels of protein in the urine that are greater than normal physiologic levels.

As used herein, the terms “diagnostic” and “prognostic” are used interchangeably, and refer to the prediction of the probable response of a subject having or developing albuminuria. As examples, the methods of the present invention provide for the detection of adiponectin presence in the blood, serum or plasma.

The terms “AMPK” or “AMP-activated protein kinase” are used interchangeably herein and refer to a regulator of the AMPK pathway. As a non-limiting example, and for reference purposes only, accession numbers for human AMPK amino acid sequences include: NP_{—}006242; NP_{—}006244; NP_{—}005390; P54619; Q9UGJ0; and NP_{—}057287 (see also International Patent application No. WO2004/050898). An “AMPK agonist” agonizes the activity of AMPK, and includes small molecules or other agents that increase the activity of the AMPK enzyme, as well as agents that specifically stimulate the expression of the AMPK polypeptide. As a non-limiting example, 5-aminoimidazole-4-carboxamide ribonucleoside (AICAR) is a cell-permeable AMPK agonist. Other AMPK agonists include, for example, metaformin, a thiazoliddineodione, AICAR and leptin.

As used herein the terms “kidney disorder” and “kidney disease” are used interchangeably, and refer to any pathological disease or condition of the kidney including, for example, those disease or disorders and conditions considered in Comprehensive Clinical Nephrology, 2^{nd} Ed, edited by Richard J Johnson and John Feehally, Mosby 2003, which is incorportated herein by reference in its entirety. In some embodiments, kidney disease may lead to hypertension or hypotension. Examples for kidney problems possibly leading to hypertension are renal artery stenosis, pyelonephritis, glomerulonephritis, kidney tumors, polycistic kidney disease, injury to the kidney, or radiation therapy affecting the kidney. Kidney disease can also be due to other disorders, for example due to obesity, diabetes, insulin resistance, or other metabolic disorders.

The terms “cardiovascular disease” and “cardiovascular disorder” as used herein refer to disorders of the heart and the vascular system, including, but not limited to congestive heart failure, myocardial infarction, ischemic diseases of the heart, all kinds of atrial and ventricular arrhythmias, hypertensive vascular diseases, peripheral vascular diseases, and atherosclerosis.

The term “subject” as used herein refers to human and non-human animals. The term “non-human animals” includes all vertebrates, e.g., mammals, such as non-human primates, (particularly higher primates), sheep, dogs, rodents (e.g. mouse or rat), guinea pigs, goats, pigs, cats, rabbits, cows, and non-mammals such as chickens, amphibians, reptiles etc. In one embodiment, the subject is human. In another embodiment, the subject is an experimental animal or animal substitute as a disease model.

The term “portion” as used herein when referring to a protein (as in “a portion of a given protein”) refers to fragments of that protein. The fragments may range in size from four amino acids residues to the entire amino acid sequence (that is, the “full size” sequence) minus one amino acid. An “active” or “functional” portion of a subject protein or polypeptide will have one or more of the biological activities of the full length protein or polypeptide. In this context, raising an immune response is not a biological activity. In one embodiment, a “portion” of adiponectin refers to the globular domain of adiponectin.

The term “effective amount” as used herein refers to the amount of therapeutic agent or pharmaceutical composition necessary or sufficient to alleviate at least one of the symptoms of the disease or disorder.

As used herein, the phrase “gene expression” is used to refer to the transcription of a gene product into mRNA and is also used to refer to the expression of the protein encoded by the gene.

As used herein, the terms “promoter” or “promoter region” or “promoter element” are used interchangeably, and refer to a segment of a nucleic acid sequence, typically but not limited to DNA or RNA or analogues thereof, that controls the transcription of the nucleic acid sequence to which it is operatively linked. The promoter region includes specific sequences that are sufficient for RNA polymerase recognition, binding and transcription initiation. This core portion of the promoter region is generally referred to as the promoter. In addition, the promoter region can include sequences which modulate this recognition, binding and transcription initiation activity of RNA polymerase. These sequences may be cis-acting or may be responsive to trans-acting factors and are included within the meaning of the term “promoter” as it is used herein. Promoters, depending upon the nature of the regulation may be constitutive or regulated.

As used herein, the term “control sequences sufficient for the expression” of, e.g., an adiponectin polypeptide refers to, at a minimum, a promoter that directs the expression of an operatively linked protein coding sequence in a cell. Where regulated expression is desired or required, control sequences sufficient for expression can further encompass cis-acting sequences, including, for example, enhancers, which often confer cell-type- or tissue-specific regulation upon a linked protein coding sequence. Alternatively, or in addition, control sequences sufficient for regulated expression can encompass inducible promoter systems responsive to the addition of an inducing agent to the system. Where necessary or desired for efficient and/or regulated expression, the term also encompasses sequences that control, e.g., transcript processing or stability, e.g., polyadenylation signals, signals that influence intracellular localization of transcripts, and AUUUA-type elements that mediate mRNA instability or other elements that influence stability or efficient translation.

The term “constitutively active promoter” refers to a promoter of a gene which is expressed at all times within a given cell. Exemplary promoters for use in mammalian cells include cytomegalovirus (CMV), and for use in prokaryotic cells include the bacteriophage T7 and T3 promoters, and the like. The term “inducible promoter” refers to a promoter of a gene which can be expressed in response to a given signal, for example addition or reduction of an agent. Non-limiting examples of an inducible promoter are “tet-on” and “tet-off” promoters, or promoters that are regulated in a specific tissue type.

The terms “operatively linked” and “operatively associated” are used interchangeably herein, and refer to the functional relationship of nucleic acid sequences with regulatory sequences, such as promoters, enhancers, transcriptional and translational stop sites, and other signal sequences. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined.

The term “agent” or “compound” as used herein and throughout the application is intended to refer to any means such as an organic or inorganic molecule, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof.

The term “biological sample” as used herein refers to a cell or population of cells or a quantity of tissue or fluid from a subject. Most often, the sample has been removed from an subject, but the term “biological sample” can also refer to cells or tissue analyzed in vivo, i.e. without removal from the subject. Often, a “biological sample” will contain cells from the animal, but the term can also refer to non-cellular biological material, such as non-cellular fractions of blood, saliva, or urine, that can be used, e.g., to measure gene expression levels. Biological samples include, but are not limited to, tissue biopsies, scrapes (e.g. buccal scrapes), whole blood, plasma, serum, urine, saliva, cell culture, or cerebrospinal fluid. Preferred biological samples include tissue biopsies and cell cultures. The sample can be obtained by removing a sample of cells from a subject, but can also be accomplished by using previously isolated cells (e.g. isolated by another person), or by performing the methods of the invention in vivo.

As used herein, the term “Nox4 inhibitor” refers to an agent that reduces the activity of the NADPH oxidase Nox4, e.g., by at least 10%, but preferably by at least 25%, 50%, 75%, 85%, 90%, 95%, 99% or more, including complete inhibition. Assays for Nox4 activity are known to those skilled in the art. Agents useful to reduce Nox4 activity include, for example, inhibitory antibodies and siRNAs directed to Nox4 RNA transcripts, as well as small molecule inhibitors of Nox4 activity. Specific Nox4 inhibitors and assays for Nox4 activity are described, e.g., in U.S. published patent application Nos. 20070037883 and 20060035358.

The term “baseline” as used herein refers to a quantitative level of a measurement, based on a level found in normal non-affected subjects. As an example, normal baseline levels of adiponectin in serum are known in the art, but are generally from 5 to 30 μg/ml in healthy humans. Levels in other sources, e.g., whole blood or a tissue sample are also known in the art or can be established by the ordinarily skilled artisan, e.g., by evaluation of levels in appropriate populations of healthy individuals.

### BEST MODES FOR PRACTICING THE INVENTION

Adiponectin protein useful in the present invention can be produced in any of a variety of methods including isolation from natural sources including tissue, production by recombinant DNA expression and purification, and the like. Adiponectin protein can also be provided “in situ” by introduction of a nucleic acid cassette containing a nucleic acid (gene) encoding the protein to the tissue of interest which then expresses the protein in the tissue.

A gene encoding adiponectin protein can be prepared by a variety of methods known in the art. For example, the gene can readily be cloned using cDNA cloning methods from any tissue expressing the protein. The accession number for the human adiponectin gene transcript is NM_{—}004797 and the rat accession number is NM_{—}144744. Protein accession numbers are NP_{—}004788 and NP_{—}653345 for human and rat respectively. See also, U.S. Pat. No. 5,869,330; US20020132773; US200230147855 and US200230176328.

The nucleotide sequences of particular use in the present invention, which encode adiponectin protein, include various DNA segments, recombinant DNA (rDNA) molecules and vectors constructed for expression of adiponectin protein. DNA molecules (segments) of this invention therefore can comprise sequences which encode whole structural genes, and fragments of structural genes encoding a protein fragment having the desired biological activity, such as promoting repair of podocyte filtration.

In one embodiment, the DNA segment is a nucleotide sequence which encodes adiponectin protein as defined herein or a biologically active fragment or portion thereof. By biologically active, it is meant that the expressed protein will have at least some of the biological activity of the intact protein found in a cell for the desired purpose. Preferably it has at least 50% of the activity, more preferably at least 75%, still more preferably at least 90% of the activity. In this context, raising an immune response is not a biological activity.

Methods to Detect Adiponectin

The present invention also provides, in other aspects, methods for detecting adiponectin in a biological sample from a subject by measuring adiponectin in a sample taken from a subject, and determining whether the level of adiponectin expression is below a baseline level in the biological sample. The various techniques, including hybridization based and amplification based methods, for measuring and evaluating adiponectin expression are described herein and known to those of skill in the art. The invention thus provides methods for detecting adiponectin expression at the RNA or protein levels wherein both results are indicative of a subject's likelihood of having or developing albuminuria.

Expression is detected in a biological sample obtained from the subject. RNA, either total RNA or mRNA, may be isolated or extracted from the biological sample. Alternatively, protein may be extracted from the biological sample. Extracted or isolated nucleic acid or protein material may be used to detection of gene expression. In one embodiment, expression of adiponectin is detected in the biological sample, for example, a blood sample. In another embodiment, blood is collected from the subject and nucleated cells are isolated from the blood, e.g. by ficoll gradient and cytospin tube use.

The methods of the present invention are also applicable to subjects who express variants of adiponectin. Determination of adiponectin expression in the biological sample may be determined by any of the methods described below, or any other method known in the art, for detection of adiponectin expression in a biological sample. The present invention encompasses methods of detecting gene expression known to those of skill in the art; see, for example, Boxer, J. Clin. Pathol. 53: 19-21 (2000). Such techniques include in situ hybridization (Stoler, Clin. Lab. Med. 12:215-36 (1990), using radioisotope or fluorophore-labeled probes; reverse transcription and polymerase chain reaction (RT-PCR); Northern blotting, dot blotting and other techniques for detecting individual genes. The probes or primers selected for gene expression evaluation are highly specific to avoid detecting closely related homologous genes. Alternatively, antibodies may be employed that recognize adiponectin antigens in various immunological assays, including immunohistochemical, western blotting, ELISA assays, etc.

In another embodiment, the methods further involve obtaining a control biological sample and detecting adiponectin expression in this control sample, such that the presence or absence of adiponectin expression in the control sample is determined. A positive control sample is useful to detect the absence or reduced expression of adiponectin expression, whereas another control sample is useful for the detection of the presence of adiponectin expression above a baseline level. For the positive control, the sample may be from the same or a different subject as the test sample, wherein the levels of adiponectin expression is known to be below a baseline, and where the sample is from the same subject, the sample may be for example from the subject before or during a particular therapeutic regime. In another control can be from the same or a different subject as the test sample, where the levels of adiponectin expression are known to be above a baseline, for example from a control sample or from a sample where adiponectin has been added.

In one embodiment, techniques that provide histological information about the biological sample are used, for example immunohistochemical or FISH-based techniques. Histological information may be used to determine that the cells expressing adiponectin can be performed. Immunohistochemical or FISH-based techniques may also be used to identify cells that express adiponectin.

Any of the following gene transcription and polypeptide or protein expression assays can be used to detect mRNA transcription and/or protein expression for adiponectin, endothelial cell marker(s), tumor endothelial cell marker(s) or any combination thereof.

Polypeptide-Based Assays

Protein or polypeptide expression, e.g. adiponectin expression, can be detected and quantified by any of a number of methods well known to those of skill in the art. Examples of analytic biochemical methods suitable for detecting adiponectin protein include electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunohistochemistry, immunocytochemistry, FACS scanning, immunoblotting, immunoprecipitation, affinity chromatography, immunoelectrophoresis, radioimmunoassay (RIA), enzyme-linked immunosorbent assays (ELISAs), immunofluorescent assays, Western blotting, and the like.

Protein expression, e.g. adiponectin expression, can be detected and quantified using various well-known immunological assays. Immunological assays refer to assays that utilize an antibody (e.g., polyclonal, monoclonal, chimeric, humanized, scFv, and fragments thereof) that specifically binds to creatine transporter polypeptide (or a fragment thereof). A number of well-established immunological assays suitable for the practice of the present invention are known, and include ELISA, radioimmunoassay (RIA), immunoprecipitation, immunofluorescence, and Western blotting.

Adiponectin antibodies (preferably anti-mammalian; more preferably anti-human), polyclonal or monoclonal, to be used in the immunological assays of the present invention are commercially available from a variety of commercial suppliers, e.g., AbCam (Cambridge UK and Cambridge, Mass.), Invitrogen Corp. (Carlsbad, Calif.), Bethyl Laboratories (Montgomery, Tex.) and Novus Biologicals (Littleton, Colo.). Alternatively, antibodies may be produced by methods well known to those skilled in the art, e.g., as described in Harlow et al., Antibodies: A Laboratory Manual, 2nd Ed; Cold Spring Harbor Laboratory Press, Cold Spring Harbor, N.Y. (1988). For example, monoclonal antibodies to adiponectin, preferably mammalian; more preferably human, can be produced by generation of hybridomas in accordance with known methods. Hybridomas formed in this manner are then screened using standard methods, such as ELISA, to identify one or more hybridomas that produce an antibody that specifically binds to the antigen of interest. Full-length antigen of interest, e.g. adiponectin, may be used as the immunogen, or, alternatively, antigenic peptide fragments of the antigen of interest may be used.

In one embodiment, levels of adiponectin can be measured from commercially available ELISA kits, by persons of ordinary skill in the art. For example but not limited to, such kits include Adiponectin kits from ALPCO (Salem, N.H., USA); R & D Systems; BioVision (Mountain View, Calif., US); Linco Research (St Charles, Md., USA); calbiochem (Germany); Gentaur (Milan, Italy) etc.

As an alternative to preparing monoclonal antibody-secreting hybridomas, a monoclonal antibody to the antigen of interest, e.g. adiponectin, may be identified and isolated by screening a recombinant combinatorial immunoglobulin library (e.g., an antibody phage display library) to thereby isolate immunoglobulin library members that bind to the antigen of interest, e.g. adiponectin. Kits for generating and screening phage display libraries are commercially available from, e.g., Dyax Corp. (Cambridge, Mass.) and Maxim Biotech (South San Francisco, Calif.). Additionally, examples of methods and reagents particularly amenable for use in generating and screening antibody display libraries can be found in the literature.

Polyclonal sera and antibodies may be produced by immunizing a suitable subject, such as a rabbit, with the antigen of choice, e.g. adiponectin, preferably mammalian; more preferably human, or an antigenic fragment thereof. The antibody titer in the immunized subject may be monitored over time by standard techniques, such as with ELISA, using immobilized marker protein. If desired, the antibody molecules directed against the antigen of interest, e.g. adiponectin, may be isolated from the subject or culture media and further purified by well-known techniques, such as protein A chromatography, to obtain an IgG fraction.

Fragments of antibodies to the antigen of interest, e.g. adiponectin, may be produced by cleavage of the antibodies in accordance with methods well known in the art. For example, immunologically active F(ab′) and F(ab′)₂ fragments may be generated by treating the antibodies with an enzyme such as pepsin. Additionally, chimeric, humanized, and single-chain antibodies to the antigen of interest, comprising both human and nonhuman portions, may be produced using standard recombinant DNA techniques. Humanized antibodies to the antigen of interest may also be produced using transgenic mice that are incapable of expressing endogenous immunoglobulin heavy and light chain genes, but which can express human heavy and light chain genes.

Antibody production is provided by the present invention. Antibodies can be prepared against the immunogen, or any portion thereof, for example a synthetic peptide based on the sequence. As stated above, antibodies are used in assays and are therefore used in determining if the appropriate enzyme has been isolated. Antibodies can also be used for removing enzymes from red cell suspensions after enzymatic conversion. Immunogens can be used to produce antibodies by standard antibody production technology well known to those skilled in the art as described generally in Harlow and Lane, Antibodies: A Laboratory Manual, Cold Springs Harbor Laboratory, Cold Spring Harbor, N.Y., 1988 and Borrebaeck, Antibody Engineering-A Practical Guide, W.H. Freeman and Co., 1992. Antibody fragments can also be prepared from the antibodies and include Fab, F(ab′).sub.2, and Fv by methods known to those skilled in the art.

In the immunological assays of the present invention, the antigen, e.g. adiponectin, is typically detected directly (i.e., the antibody to the antigen of interest is labeled) or indirectly (i.e., a secondary antibody that recognizes the antibody to the antigen of interest is labeled) using a detectable label. The particular label or detectable group used in the assay is usually not critical, as long as it does not significantly interfere with the specific binding of the antibodies used in the assay.

The immunological assays of the present invention may be competitive or noncompetitive. In competitive assays, the amount of adiponectin in a sample is measured indirectly by measuring the amount of added (exogenous) adiponectin displaced from a capture agent, i.e. an anti-adiponectin antibody, by the adiponectin in the sample. In noncompetitive assays, the amount of adiponectin in a sample is directly measured. In a preferred noncompetitive “sandwich” assay, the capture agent (e.g., a first antibody) is bound directly to a solid support (e.g., membrane, microtiter plate, test tube, dipstick, glass or plastic bead) where it is immobilized. The immobilized agent then captures any antigen of interest present in the sample. The immobilized antigen of interest can then be detected using a second labeled antibody to the antigen of interest. Alternatively, the second antibody can be detected using a labeled secondary antibody that recognizes the second antibody.

A preferred method of measuring the expression of the antigen of interest, e.g. adiponectin, is by antibody staining with an antibody that binds specifically to the antigen employing a labeling strategy that makes use of luminescence or fluorescence. Such staining may be carried out on fixed tissue or cells that are ultimately viewed and analyzed under a microscope. Staining carried out in this manner can be scored visually or by using optical density measurements. Staining may also be carried out using either live or fixed whole cells in solution, e.g. cells isolated from blood. In some embodiments, such cells can be analyzed using a fluorescence activated cell sorter (FACS), which can determine both the number of cells stained and the intensity of the luminescence or fluorescence. Such techniques are well known in the art, and exemplary techniques are described in Luwor et al. ((2001), Cancer Res. 61:5355-61). One of skill in the art will realize that other techniques of detecting expression might be more or less sensitive than these techniques. As meant herein, cells express little or no antigen if little or no antigen can be detected using an antibody staining technique that relies on luminescence or fluorescence.

Alternatively, adiponectin expression can be detected in vivo in a subject by introducing into the subject a labeled antibody to the adiponectin protein. For example, the antibody can be labeled with a radioactive marker whose presence and location in a subject can be detected by standard imaging techniques.

In one preferred embodiment, immunohistochemistry (“IHC”) and immunocytochemistry (“ICC”) techniques, for example, may be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of a specific antibody, wherein antibodies are used to specifically target molecules inside or on the surface of cells. The antibody typically contains a marker that will undergo a biochemical reaction, and thereby experience a change color, upon encountering the targeted molecules. In some instances, signal amplification may be integrated into the particular protocol, wherein a secondary antibody, that includes the marker stain, follows the application of a primary specific antibody.

Immunoshistochemical assays are known to those of skill in the art (e.g., see Jalkanen, et al., J. Cell. Biol. 101:976-985 (1985); Jalkanen, et al., J. Cell. Biol. 105:3087-3096 (1987). Typically, for immunohistochemistry, tissue sections are obtained from a patient and fixed by a suitable fixing agent such as alcohol, acetone, and paraformaldehyde, to which is reacted an antibody. Conventional methods for immunohistochemistry are described in Harlow and Lane (eds) (1988) In “Antibodies A Laboratory Manual”, Cold Spring Harbor Press, Cold Spring Harbor, N.Y.; Ausbel et al (eds) (1987), in Current Protocols In Molecular Biology, John Wiley and Sons (New York, N.Y.). Biological samples appropriate for such detection assays include, but are not limited to, cells, tissue biopsy, whole blood, plasma, serum, sputum, cerebrospinal fluid, breast aspirates, pleural fluid, urine and the like.

For direct labeling techniques, a labeled antibody is utilized. For indirect labeling techniques, the sample is further reacted with a labeled substance.

Alternatively, immunocytochemistry may be utilized. In general, cells are obtained from a patient and fixed by a suitable fixing agent such as alcohol, acetone, and paraformaldehyde, to which is reacted an antibody. Methods of immunocytological staining of human samples is known to those of skill in the art and described, for example, in Brauer et al., 2001 (FASEB J, 15, 2689-2701), Smith-Swintosky et al., 1997. Immunological methods of the present invention are advantageous because they require only small quantities of biological material. Such methods may be done at the cellular level and thereby necessitate a minimum of one cell.

In one embodiment, peripheral blood of the subject is used for immuno-based methods to detect adiponectin. Blood may be purified to isolate nucleated cells, e.g. by Ficoll gradient and cytospin tubes. The nucleated cells may be analyzed by antibodies to adiponectin, e.g. fluorescently-tagged antibodies, antibodies bound to fluorescently tagged antibodies, that are detected by a fluorescence activated cell sorter (FACS).

Amplification-Based Assays

In one embodiment, amplification-based assays can be used to detect, and optionally quantify, adiponectin expression. In such amplification-based assays, the adiponectin mRNA in the sample obtained from the subject act as template(s) in an amplification reaction carried out with a nucleic acid primer that contains a detectable label or component of a labeling system. Suitable amplification methods include, but are not limited to, polymerase chain reaction (PCR); reverse-transcription PCR (RT-PCR); ligase chain reaction (LCR) (see Wu and Wallace (1989) Genomics 4: 560, Landegren et al. (1988) Science 241: 1077, and Barringer et al. (1990) Gene 89: 117; transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874); dot PCR, and linker adapter PCR, etc. The known nucleic acid sequence for adiponectin (Accession No.: NM_{—}004797; NP_{—}653345. See also, U.S. Pat. No. 5,869,330; US20020132773; US200230147855 and US200230176328) is sufficient to enable one of skill to routinely select primers to amplify any portion of the gene.

PCR-Based Gene Expression Detection Methods

Reverse Transcriptase PCR (RT-PCR)

One of the most sensitive and most flexible PCR-based gene expression detection methods is RT-PCR, which can be used to determine presence or absence of expression and also to quantitate levels of gene expression.

The first step is the isolation of mRNA from a target sample. The starting material is typically total RNA isolated from tissue samples, for example but not limited to, blood, plasma, adiopocyte cells, podocytes, mRNA can be extracted, for example, from frozen or archived paraffin-embedded and fixed, e.g. formalin-fixed, tissue samples.

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andrs et al., BioTechniques 18:42044 (1995). In particular, RNA isolation can be performed using purification kit, buffer set and protease from commercial manufacturers, such as Qiagen (Valencia, Calif.), according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc., Austin, Tex.). Total RNA from tissue samples can be isolated using RNA Stat-60 (Tel-Test, Friendswood, Tex.). RNA prepared from tumor can be isolated, for example, by cesium chloride density gradient centrifugation.

As RNA cannot serve as a template for PCR, the first step in gene expression detection by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction. Methods for reverse transcription of template RNA to cDNA are well known to persons skilled in the art, and are encompassed in the methods of this invention.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a relatively constant level among different tissues, and is unaffected by the experimental treatment. RNAs frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

Real-time PCR can be performed, for example, using a Perkin Elmer/Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, beta-actin) primers and probes may be obtained commercially from, for example, Perkin Elmer/Applied Biosystems (Foster City, Calif.). To quantitate the amount of the specific nucleic acid of interest in a sample, a standard curve is generated using a control. Standard curves may be generated using the Ct values determined in the real-time PCR, which are related to the initial concentration of the nucleic acid of interest used in the assay. Standard dilutions ranging from 10-10⁶ copies of the gene of interest are generally sufficient. In addition, a standard curve is generated for the control sequence. This permits standardization of initial content of the nucleic acid of interest in a tissue sample to the amount of control for comparison purposes.

Methods of real-time quantitative PCR using TaqMan probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994.

MassARRAY System

In the MassARRAY-based gene expression profiling method, developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059-3064 (2003).

Other PCR-Based Methods

Further PCR-based techniques include, for example, differential display (Liang and Pardee, Science 257:967-971 (1992)); amplified fragment length polymorphism (iAFLP) (Kawamoto et al., Genome Res. 12:1305-1312 (1999)); BeadArray™. technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminexl-00 LabMAP system and multiple color-coded microspheres (Luminex Corp., Austin, Tex.) in a rapid assay for gene expression (Yang et al., Genome Res. 11:1888-1898 (2001)); and high coverage expression profiling (HiCEP) analysis (Fukumura et al., Nucl. Acids. Res. 31(16) e94 (2003).

Other suitable amplification methods include, but are not limited to ligase chain reaction (LCR) (see Wu and Wallace (1989) Genomics 4:560, Landegren et al. (1988) Science 241:1077, and Barringer et al. (1990) Gene 89:117), transcription amplification (Kwoh et al. (1989) Proc. Natl. Acad. Sci. USA 86:1173), self-sustained sequence replication (Guatelli et al. (1990) Proc. Nat. Acad. Sci. USA 87:1874), dot PCR, and linker adapter PCR, etc.

Hybridization-Based Assays

Hybridization assays can be used to detect adipontein transcription. Hybridization-based assays include, but are not limited to, methods such as Northern blots or RNA in situ hybridization, e.g. fluorescent in situ hybridization (FISH). The methods can be used in a wide variety of formats including, but not limited to substrate, e.g. membrane or glass, bound methods or array-based approaches as described below.

Nucleic acid hybridization simply involves contacting a nucleic acid probe with sample polynucleotides under conditions where the probe and its complementary target nucleotide sequence can form stable hybrid duplexes through complementary base pairing. The nucleic acids that do not form hybrid duplexes are then washed away leaving the hybridized nucleic acids to be detected, typically through detection of an attached detectable label or component of a labeling system. Methods of detecting and/or quantifying polynucleotides using nucleic acid hybridization techniques are known to those of skill in the art (see Sambrook et al. supra). Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587. Methods of optimizing hybridization conditions are described, e.g., in Tijssen Laboratory Techniques in Biochemistry and Molecular Biology, Vol. 24: Hybridization With Nucleic Acid Probes, Elsevier, N.Y.).

The nucleic acid probes used herein for detection of adiponecitin mRNA can be full-length or less than the full-length of the adiponectin transcript. Shorter probes are generally empirically tested for specificity. Preferably, nucleic acid probes are at least about 15, and more preferably about 20 bases or longer, in length. (See Sambrook et al. for methods of selecting nucleic acid probe sequences for use in nucleic acid hybridization.) Visualization of the hybridized probes allows the qualitative determination of the presence or absence of the channel subunit mRNA of interest, and standard methods (such as, e.g., densitometry where the nucleic acid probe is radioactively labeled) can be used to quantify the level of adiponectin expression.

A variety of additional nucleic acid hybridization formats are known to those skilled in the art. Standard formats include sandwich assays and competition or displacement assays. Sandwich assays are commercially useful hybridization assays for detecting or isolating polynucleotides. Such assays utilize a “capture” nucleic acid covalently immobilized to a solid support and a labeled “signal” nucleic acid in solution. The sample provides the target polynucleotide. The capture nucleic acid and signal nucleic acid each hybridize with the target polynucleotide to form a “sandwich” hybridization complex.

Northern Blot

One method for evaluating adiponectin transcription in a sample involves a Northern transfer. Methods for doing Northern Blots are known to those of skill in the art (see Current Protocols in Molecular Biology, Ausubel, et al., Eds., Greene Publishing and Wiley-Interscience, New York, 1995, or Sambrook et al., Molecular Cloning: A Laboratory Manual, 2d Ed. vol. 1-3, Cold Spring Harbor Press, NY, 1989). In such an assay, the total RNA or polyA RNA (typically fragmented and separated on an electrophoretic gel) is hybridized to a probe specific for the target region.

Fluorescence In Situ Hybridization (FISH)

In another embodiment, RNA-FISH is used to determine adiponectin transcription in a sample. Fluorescence in situ hybridization (FISH) is known to those of skill in the art (see Angerer, 1987 Meth. Enzymol., 152: 649). Generally, in situ hybridization comprises the following major steps: (1) fixation of tissue or biological structure to be analyzed; (2) prehybridization treatment of the biological structure to increase accessibility of target RNA, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments.

In a typical in situ hybridization assay, cells or tissue sections are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets, e.g., cells, are then typically washed at a predetermined stringency or at an increasing stringency until an appropriate signal to noise ratio is obtained.

The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions.

In some applications it is necessary to block the hybridization capacity of repetitive sequences. Thus, in some embodiments, tRNA, human genomic DNA, salmon sperm DNA or Cot-1 DNA is used to block non-specific hybridization.

Thus, in one embodiment of the present invention, the presence or absence of adiponectin expression is determined by RNA-FISH. In one embodiment, adiponectin expression is determined in cells isolated from blood.

Microarray Based Expression Analysis

In one embodiment, the methods of the invention can be utilized in array-based hybridization formats for the detection of adiponectin in the biological sample. In an array format, a large number of different hybridization reactions can be run essentially “in parallel.” This provides rapid, essentially simultaneous, evaluation of a number of hybridizations in a single experiment. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays, can be produced according to a wide variety of methods well known to those of skill in the art. For example, in a simple embodiment, “low-density” arrays can simply be produced by spotting (e.g. by hand using a pipette) different nucleic acids at different locations on a solid support (e.g. a glass surface, a membrane, etc.). This simple spotting approach has been automated to produce high-density spotted microarrays. For example, U.S. Pat. No. 5,807,522 describes the use of an automated system that taps a microcapillary against a surface to deposit a small volume of a biological sample. The process is repeated to generate high-density arrays. Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high-density oligonucleotide microarrays. Synthesis of high-density arrays is also described in U.S. Pat. Nos. 5,744,305; 5,800,992; and 5,445,934.

Hybridization assays according to the invention can also be carried out using a MicroElectroMechanical System (MEMS), such as the Protiveris' multicantilever array.

Adiponectin mRNA is detected in the above-described polynucleotide-based assays by means of a detectable label. Any of the labels discussed above can be used in the polynucleotide-based assays of the invention. The label may be added to a probe or primer or sample polynucleotides prior to, or after, the hybridization or amplification. So called “direct labels” are detectable labels that are directly attached to or incorporated into the labeled polynucleotide prior to conducting the assay. In contrast, so called “indirect labels” are joined to the hybrid duplex after hybridization. In indirect labeling, one of the polynucleotides in the hybrid duplex carries a component to which the detectable label binds. Thus, for example, a probe or primer can be biotinylated before hybridization. After hybridization, an avidin-conjugated fluorophore can bind the biotin-bearing hybrid duplexes, providing a label that is easily detected. For a detailed review of methods of the labeling and detection of polynucleotides, see Laboratory Techniques in Biochemistry and Molecular Biology, Vol. 24: Hybridization With Nucleic Acid Probes, P. Tijssen, ed. Elsevier, N.Y., (1993).

In an alternative embodiment of the present invention, adiponectin mRNA expression is analyzed via microarray-based platforms. Microarray technology offers high resolution. Details of various microarray methods can be found in the literature. See, for example, U.S. Pat. No. 6,232,068; Pollack et al., Nat. Genet., 23(1):41-6, (1999), Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211 and others.

Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In Situ Hybridization Protocols, Choo, ed., Humana Press, Totowa, N.J. (1994), Pinkel et al. (1998) Nature Genetics 20: 207-211, or of Kallioniemi (1992) Proc. Natl. Acad Sci USA 89:5321-5325 (1992), etc.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

The sensitivity of the hybridization assays can be enhanced through use of a polynucleotide amplification system that multiplies the target polynucleotide being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

Detection and quantification of gene expression, e.g. adiponectin expression, may be carried out through direct hybridization based assays or amplification based assays. The hybridization based techniques for measuring gene transcript are known to those skilled in the art (Sambrook et al., Molecular Cloning: A Laboratory Manual, 2d Ed. vol. 1-3, Cold Spring Harbor Press, NY, 1989). For example, one method for evaluating the presence, absence, or quantity of adiponectin gene expression is by Northern blot. Isolated mRNAs from a given biological subject are electrophoresed to separate the mRNA species, and transferred from the gel to a membrane, for example, a nitrocellulose or nylon filter. Labeled adiponectin probes are then hybridized to the membrane to identify and quantify the respective mRNAs. An example of amplification based assays include RT-PCR, which is well known in the art (Ausubel et al., Current Protocols in Molecular Biology, eds. 1995 supplement). In a preferred embodiment, quantitative RT-PCR is used to allow the numerical comparison of the level of respective adiponectin mRNAs in different samples. A Real-Time or TaqMan-based assay also can be used to adiponectin gene transcription.

Other Diagnostic Methods

In some embodiments, the expression of adiponectin below a baseline may be due to a mutation in adiponectin. An agent for detecting mutant adiponectin protein is an antibody capable of binding to mutant adiponectin, preferably an antibody with a detectable label. Antibodies can be polyclonal, or more preferably, monoclonal. An intact antibody, or a fragment thereof (e.g., F_{ab} or F(ab)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect mutant adiponectin mRNA, protein, or genomic DNA in a biological sample in vitro as well as in vivo. For example, in vitro techniques for detection of mutant adiponectin mRNA include Northern hybridizations and in situ hybridizations. In vitro techniques for detection of mutant adiponectin protein include enzyme linked immunosorbent assays (ELISAs), Western blots, immunoprecipitations, and immunofluorescence. In vitro techniques for detection of mutant adiponectin genomic DNA include Southern hybridizations. Furthermore, in vivo techniques for detection of mutant adiponectin protein include introducing into a subject a labeled anti-mutant adiponectin protein antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject.

In another embodiment, the methods further involve obtaining a control biological sample from a control subject, contacting the control sample with a compound or agent capable of detecting mutant adiponectin protein, mRNA, or genomic DNA, such that the presence of mutant adiponectin protein, mRNA or genomic DNA is detected in the biological sample, and comparing the presence of mutant adiponectin protein, mRNA or genomic DNA in the control sample with the presence of mutant adiponectin protein, mRNA or genomic DNA in the test sample.

Method of Treating a Subject

In one aspect, the present invention provides for a method for decreasing the risk of developing or reducing the effects of albuminuria in a subject. In one embodiment, the subject can be a mammal. In another embodiment, the mammal can be a human, although the invention is effective with respect to all mammals. The method comprises administering to the subject an effective amount of a pharmaceutical composition comprising adiponectin protein, or portion thereof, in a pharmaceutically acceptable carrier. Alternatively, a pharmaceutical composition comprising a nucleic acid encoding adiponectin, or portion thereof, can be administered.

In one embodiment, the adiponectin is globular adiponectin (gAd). In another embodiment, different oligimeric forms of adiponectin are used, for example trimeric or hexameric protein.

In an important embodiment of the invention, subjects with obesity and low levels of adiponectin are administered an effective amount of a pharmaceutical composition comprising adiponectin protein, or portion thereof, or a nucleic acid encoding adiponectin in a pharmaceutically acceptable carrier.

Mimetics of adiponectin also can be used in accordance with the present invention to modulate podocyte function. The design of mimetics is known to those skilled in the art, and is generally understood to be peptides or other relatively small molecules that have an activity the same or similar to that of a larger molecule, often a protein, on which they are modeled.

Variations and modifications to the above protein and vectors can be used to increase or decrease adiponectin expression, and to provide means for targeting. For example, adiponectin can be linked with a molecular counter-ligand for podocytes, for example but not limited to nephrin molecules, such as nephrin-adiponectin, to make these agents tissue specific.

In one embodiment, the protein or fragment thereof is linked to a carrier to enhance its bioavailability. Such carriers are known in the art and include poly (alkyl) glycol such as poly ethylene glycol (PEG). Fusion to serum albumin can also increase the serum half-life of therapeutic polypeptides.

Similarly, techniques for making small oligopeptides and polypeptides that exhibit activity of larger proteins from which they are derived (in primary sequence) are well known and have become routine in the art. Thus, peptide analogs of proteins of the invention, such as peptide analogs of adiponectin that exhibit agonist activity also are useful in the invention.

The dosage ranges for the administration of adiponectin protein depend upon the form of the protein, and its potency, as described further herein, and are amounts large enough to produce the desired effect in which the effects of albuminuria are reduced, for example but not limited to; decreased podocyte permeability; restoration of glomerular podocyte foot morphology; decreased albumin in urine; restoration of urine albumin/creatinine ratios; and decreased renal failure. The dosage should not be so large as to cause adverse side effects. Generally, the dosage will vary with the age, condition, and sex of the patient and can be determined by one of skill in the art. The dosage can also be adjusted by the individual physician in the event of any complication. Typically, the dosage ranges from 0.001 mg/kg body weight to 0.5 mg/kg body weight. In one embodiment, the dose range is from 5 μg/kg body weight to 30 μg/kg body weight. The doses can be given once a day, less than once a day or multiple times a day in order to achieve a therapeutically effective

Dose.

A therapeutically effective amount is an amount of adiponectin protein, or nucleic acid encoding for adiponectin, that is sufficient to produce a statistically significant, measurable change in albuminuria. As an example, an amount of an agent that reduces albuminuria in a subject by at least 5% or more (preferably at least 10%, 25%, 30% or more) is considered therapeutically effective. Such effective amounts can be gauged in clinical trials as well as animal studies, for example using the adiponectin knockout mouse. In some embodiments, the effect on albuminuria can be determined by assaying urine albumin/creatinine ratios from timed overnight collections and measurement of the adipocytokine, adiponectin, IL6 and PAI-1 levels by methods known to one skilled in the art.

In another embodiment, methods to increase adiponectin levels are also encompassed in the invention. Such method include, but are not limited to; weight reduction; renin-angiotensin system blockade (38); and PPAR-gamma agonists (39). In one embodiment, the subject may be administered a therapeutically effective amount of metformin (also known as trade names; Diabex®, Diaformin, Glucophage, Fortamet, Riomet, Glumetza) or metaformin hydrorchloride or analogues or mimetics thereof, as metformin raises AMPK activity independent of adiponectin (40).

The adiponectin protein or nucleic acid vector expressing such protein can be administered parenterally by injection or by gradual infusion over time. Although the tissue to be treated can typically be accessed in the body by systemic administration and therefore most often treated by intravenous administration of therapeutic compositions, other tissues and delivery means are contemplated. Thus, compositions of the invention can be administered intravenously, intraperitoneally, intramuscularly, subcutaneously, intracavity, transdemmally, and can be delivered by peristaltic means, if desired, or by other means known by those skilled in the art.

The therapeutic compositions containing adiponectin protein or nucleic acid vector expressing the protein can be conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

Adiponectin protein and vectors may be adapted for catheter-based delivery systems including coated balloons, slow-release drug-eluting stents, microencapsulated PEG liposomes, or nanobeads for delivery using direct mechanical intervention with or without adjunctive techniques such as ultrasound.

In some embodiments, the adiponectin protein of the invention may be combined with a therapeutically effective amount of another albuminuria agent. In addition, the adiponectin protein of the invention may further be combined with a therapeutically effective amount another agent known to be effective at treating other disorders; for example but not limited to kidney disorders; cardiovascular disorders; obesity disorders etc.

Gene Therapy

The invention also includes a recombinant DNA molecule (rDNA) comprising a DNA segment encoding an adiponectin polypeptide as described herein. An expressible rDNA can be produced by operatively linking a promoter to an adiponectin encoding DNA segment of the present invention, creating a cassette. The cassette can be administered by any known means including catheter, vector, gene gun, etc.

In one embodiment the DNA segment codes for an amino acid residue sequence substantially the same as, and preferably consisting essentially of, an amino acid residue sequence or portions thereof corresponding to human adiponectin protein described herein.

A nucleic acid is any polynucleotide or nucleic acid fragment, whether it be a polyribonucleotide of polydeoxyribonucleotide, i.e., RNA or DNA, or analogs thereof such as PNA. DNA segments are produced by a number of means including chemical synthesis methods and recombinant approaches, preferably by cloning or by polymerase chain reaction (PCR).

The adiponectin gene of this invention can be cloned from a suitable source of genomic DNA or messenger RNA (mRNA) by a variety of biochemical methods. Cloning these genes can be conducted according to the general methods known in the art. Sources of nucleic acids for cloning an adiponectin gene suitable for use in the methods of this invention can include genomic DNA or messenger RNA (mRNA) in the form of a cDNA library, from a tissue believed to express these proteins.

A preferred cloning method involves the preparation of a cDNA library using standard methods, and isolating the adiponectin-encoding or nucleotide sequence by PCR amplification using paired oligonucleotide primers based on nucleotide sequences described herein. Alternatively, the desired cDNA clones can be identified and isolated from a cDNA or genomic library by conventional nucleic acid hybridization methods using a hybridization probe based on the nucleic acid sequences described herein. Other methods of isolating and cloning suitable adiponectin-encoding nucleic acids are readily apparent to one skilled in the art.

The choice of promoters to which a DNA segment of the present invention is operatively linked depends directly, as is well known in the art, on the functional properties desired, e.g., protein expression, and the host cell to be transformed. Promoters that express in prokaryotic and eukaryotic systems are familiar to one of ordinary skill in the art, and are described by Sambrook et al., Molecular Cloning: A Laboratory Manual Cold Spring Harbor Laboratory (2001). In one embodiment, the promoter is an inducible promoter. In another embodiment, the promoter is a nucleic acid sequence which specifically induces the expression of the gene (for example, adiponectin) to which it is operatively linked to be expressed specifically in the kidney tissue. In such an embodiment, the promoter is an kidney-specific promoter.

Expression vectors compatible with eukaryotic cells, preferably those compatible with vertebrate cells, can be used to form the recombinant DNA molecules of the present invention. Eukaryotic cell expression vectors are well known in the art and are available from several commercial sources. Typically, such vectors are provided containing convenient restriction sites for insertion of the desired DNA segment. These vectors can be viral vectors such as adenovirus, adeno-associated virus, pox virus such as an orthopox (vaccinia and attenuated vaccinia), avipox, lentivirus, murine moloney leukemia virus, etc.

Additionally, a nucleotide sequence that encodes adiponectin, or biologically active fragment thereof, can also be delivered using other means. Such gene transfer methods for gene therapy fall into three broad categories: (1) physical (e.g., electroporation, direct gene transfer and particle bombardment), (2) chemical (e.g. lipid-based carriers and other non-viral vectors) and (3) biological (e.g. virus derived vectors). For example, non-viral vectors such as liposomes coated with DNA may be directly injected intravenously into the patient. It is believed that the liposome/DNA complexes are concentrated in the liver where they deliver the DNA to macrophages and Kupffer cells.

Gene therapy methodologies can also be described by delivery site, for example directly to the adrenal gland. Fundamental ways to deliver genes include ex vivo gene transfer, in vivo gene transfer, and in vitro gene transfer. In ex vivo gene transfer, cells are taken from the patient and grown in cell culture. The DNA is transfected into the cells, the transfected cells are expanded in number and then reimplanted in the patient. In in vitro gene transfer, the transformed cells are cells growing in culture, such as tissue culture cells, and not particular cells from a particular patient. These “laboratory cells” are transfected, the transfected cells are selected and expanded for either implantation into a patient or for other uses. In vivo gene transfer involves introducing the DNA into the cells of the patient when the cells are within the patient. All three of the broad based categories described above may be used to achieve gene transfer in vivo, ex vivo, and in vitro.

Mechanical (i.e. physical) methods of DNA delivery can be achieved by direct injection of DNA, such as catheters, preferably a catheter containing the cassette in a suitable carrier, microinjection of DNA into germ or somatic cells, pneumatically delivered DNA-coated particles, such as the gold particles used in a “gene gun,” and inorganic chemical approaches such as calcium phosphate transfection. It has been found that physical injection of plasmid DNA into muscle cells yields a high percentage of cells which are transfected and have a sustained expression of marker genes. The plasmid DNA may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylactic use. The DNA could be reinjected periodically to maintain the gene product level without mutations occurring in the genomes of the recipient cells. Non-integration of exogenous DNAs may allow for the presence of several different exogenous DNA constructs within one cell with all of the constructs expressing various gene products.

Particle-mediated gene transfer may also be employed for injecting DNA into cells, tissues and organs. With a particle bombardment device, or “gene gun,” a motive force is generated to accelerate DNA-coated high density particles (such as gold or tungsten) to a high velocity that allows penetration of the target organs, tissues or cells. Electroporation for gene transfer uses an electrical current to make cells or tissues susceptible to electroporation-mediated gene transfer. A brief electric impulse with a given field strength is used to increase the permeability of a membrane in such a way that DNA molecules can penetrate into the cells. The techniques of particle-mediated gene transfer and electroporation are well known to those of ordinary skill in the art.

Chemical methods of gene therapy involve carrier mediated gene transfer through the use of fusogenic lipid vesicles such as liposomes or other vesicles for membrane fusion. A carrier harboring a DNA of interest can be conveniently introduced into body fluids or the bloodstream and then site specifically directed to the target organ or tissue in the body. Liposomes, for example, can be developed which are cell specific or organ specific. The foreign DNA carried by the liposome thus will be taken up by those specific cells. Injection of immunoliposomes that are targeted to a specific receptor on certain cells can be used as a convenient method of inserting the DNA into the cells bearing the receptor. Another carrier system that has been used is the asialoglycoprotein/polylysine conjugate system for carrying DNA to hepatocytes for in vivo gene transfer.

Transfected DNA may also be complexed with other kinds of carriers so that the DNA is carried to the recipient cell and then resides in the cytoplasm or in the nucleoplasm of the recipient cell. DNA can be coupled to carrier nuclear proteins in specifically engineered vesicle complexes and carried directly into the nucleus.

Carrier mediated gene transfer may also involve the use of lipid-based proteins which are not liposomes. For example, lipofectins and cytofectins are lipid-based positive ions that bind to negatively charged DNA, forming a complex that can ferry the DNA across a cell membrane. Fectins may also be used. Another method of carrier mediated gene transfer involves receptor-based endocytosis. In this method, a ligand (specific to a cell surface receptor) is made to form a complex with a gene of interest and then injected into the bloodstream; target cells that have the cell surface receptor will specifically bind the ligand and transport the ligand-DNA complex into the cell.

Biological gene therapy methodologies usually employ viral vectors to insert genes into cells. The term “vector” as used herein in the context of biological gene therapy means a carrier that can contain or associate with specific polynucleotide sequences and which functions to transport the specific polynucleotide sequences into a cell. The transfected cells may be cells derived from the patient's normal tissue, the patient's diseased tissue, or may be non-patient cells. Examples of vectors include liposomes, and lipid-DNA complexes discussed above, plasmids and infective microorganisms such as viruses, or non-viral vectors such as the ligand-DNA conjugates (preferably the ligand is to a receptor preferentially expressed on the cell of interest. In one embodiment, one uses an antibody as the ligand.)

Viral vector systems which may be utilized in the present invention include, but are not limited to, (a) adenovirus vectors; (b) retrovirus vectors; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. In the preferred embodiment the vector is an adenovirus.

Thus, a wide variety of gene transfer/gene therapy vectors and constructs are known in the art. These vectors are readily adapted for use in the methods of the present invention. By the appropriate manipulation using recombinant DNA/molecular biology techniques to insert an operatively linked adiponectin encoding nucleic acid segment into the selected expression/delivery vector, many equivalent vectors for the practice of the present invention can be generated.

It will be appreciated by those of skill that cloned genes readily can be manipulated to alter the amino acid sequence of a protein. The cloned gene for adiponectin can be manipulated by a variety of well known techniques for in vitro mutagenesis, among others, to produce variants of the naturally occurring human protein, herein referred to as muteins or variants or mutants of adiponectin, which may be used in accordance with the invention.

The variation in primary structure of muteins of adiponectin useful in the invention, for instance, may include deletions, additions and substitutions. The substitutions may be conservative or non-conservative. The differences between the natural protein and the mutein generally conserve desired properties, mitigate or eliminate undesired properties and add desired or new properties.

Pharmaceutical Compositions

The present invention provides therapeutic compositions useful for practicing the therapeutic methods described herein. Therapeutic compositions of the present invention contain a physiologically tolerable carrier together with adiponectin protein or vector capable of expressing adiponectin protein as described herein, dissolved or dispersed therein as an active ingredient. In a preferred embodiment, the therapeutic composition is not immunogenic when administered to a mammal or human patient for therapeutic purposes.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to a protein or polypeptide with which it is admixed.

The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary of such additional liquid phases are glycerin, vegetable oils such as cottonseed oil, and water-oil emulsions.

For topical application, the carrier may in the form of, for example, and not by way of limitation, an ointment, cream, gel, paste, foam, aerosol, suppository, pad or gelled stick.

The amount of the active adiponectin protein (referred to as “agents”) used in the invention that will be effective in the treatment of a particular disorder or condition will depend on the nature of the disorder or condition, and can be determined by standard clinical techniques. In addition, assays such as those discussed in the examples section may optionally be employed to help identify optimal dosage ranges.

The precise dose to be employed in the formulation will also depend on the route of administration, and the seriousness of the disease or disorder, and should be decided according to the judgment of the practitioner and each subject's circumstances. Suitable dosage ranges for administration of agents are generally about 0.001 mg/kg body weight to 0.5 mg/kg body weight. In some embodiments, the suitable range for administration is 5 μg/kg body weight to 30 μg/kg body weight. Effective doses may be extrapolated from dose-response curves derived from in vitro or animal model test bioassays or systems.

Administration of the doses recited above can be repeated for a limited period of time. In some embodiments, the doses are given once a day, or multiple doses a day, for example but not limited to three times a day. In a preferred embodiment, the doses recited above are administered daily for several weeks or months. The duration of treatment depends upon the subject's clinical progress and responsiveness to therapy.

The route of administration can be any route known to persons skilled in the art, for example but not limited to parenteral, including intravenous and intraarterial administration, intrathecal administration, intraventricular administration, intraparenchymal, intracranial, intracistemal, intrastriatal, and intranigral administration.

The invention also contemplates an article of manufacture which is a labeled container for providing adiponectin protein of the invention. An article of manufacture comprises packaging material and a pharmaceutical agent contained within the packaging material.

The pharmaceutical agent in an article of manufacture is any of the compositions of the present invention suitable for providing adiponectin protein and formulated into a pharmaceutically acceptable form as described herein according to the disclosed indications. Thus, the composition can comprise adiponectin protein or a DNA molecule which is capable of expressing the protein.

The article of manufacture contains an amount of pharmaceutical agent sufficient for use in treating a condition indicated herein, either in unit or multiple dosages. The packaging material comprises a label which indicates the use of the pharmaceutical agent contained therein, e.g., for the treatment or reduction of the risk of albuminuria, and the albuminuria-like conditions or conditions and disorders where there the pathology is associated with the reduction in adiponectin.

The label can further include instructions for use and related information as may be required for marketing. The packaging material can include container(s) for storage of the pharmaceutical agent.

As used herein, the term packaging material refers to a material such as glass, plastic, paper, foil, and the like capable of holding within fixed means a pharmaceutical agent. Thus, for example, the packaging material can be plastic or glass vials, laminated envelopes and the like containers used to contain a pharmaceutical composition including the pharmaceutical agent.

In preferred embodiments, the packaging material includes a label that is a tangible expression describing the contents of the article of manufacture and the use of the pharmaceutical agent contained therein.

Screening for Adiponectin Agonists

Also provided herein are methods of screening for agonists of adiponectin activity or expression.

Test Compounds for Screening Targeting Agents

In the methods of the present invention, a variety of test compounds and physical conditions from various sources can be screened for the ability of the compound to target adiponectin and/or target AMPK. Method to screen for AMPK agonists are outlined in Patent Application WO2004/050898, incorporated herein on its entirety for reference, and are encompassed for use with this invention.

Compounds to be screened can be naturally occurring or synthetic molecules. Compounds to be screened can also be obtained from natural sources, such as, marine microorganisms, algae, plants, and fungi. The test compounds can also be minerals or oligo agents. Alternatively, test compounds can be obtained from combinatorial libraries of agents, including peptides or small molecules, or from existing repertories of chemical compounds synthesized in industry, e.g., by the chemical, pharmaceutical, environmental, agricultural, marine, cosmetic, drug, and biotechnological industries. Test compounds can include, e.g., pharmaceuticals, therapeutics, agricultural or industrial agents, environmental pollutants, cosmetics, drugs, organic and inorganic compounds, lipids, glucocorticoids, antibiotics, peptides, proteins, sugars, carbohydrates, chimeric molecules, and combinations thereof.

Combinatorial libraries can be produced for many types of compounds that can be synthesized in a step-by-step fashion. Such compounds include polypeptides, proteins, nucleic acids, beta-turn mimetics, polysaccharides, phospholipids, hormones, prostaglandins, steroids, aromatic compounds, heterocyclic compounds, benzodiazepines, oligomeric N-substituted glycines and oligocarbamates. In the method of the present invention, the preferred test compound is a small molecule, nucleic acid and modified nucleic acids, peptide, peptidomimetic, protein, glycoprotein, carbohydrate, lipid, or glycolipid. In certain embodiments, the nucleic acid is DNA or RNA.

Large combinatorial libraries of compounds can be constructed by the encoded synthetic libraries (ESL) method described in Affymax, WO 95/12608, Affymax WO 93/06121, Columbia University, WO 94/08051, Pharmacopeia, WO 95/35503 and Scripps, WO 95/30642 (each of which is incorporated herein by reference in its entirety for all purposes). Peptide libraries can also be generated by phage display methods. See, e.g., Devlin, WO 91/18980. Compounds to be screened can also be obtained from governmental or private sources, including, e.g., the DIVERSet E library (16,320 compounds) from ChemBridge Corporation (San Diego, Calif.), the National Cancer Institute's (NCI) Natural Product Repository, Bethesda, Md., the NCI Open Synthetic Compound Collection, Bethesda, Md., NCI's Developmental Therapeutics Program, or the like.

Additionally, natural and synthetically produced libraries and compounds are readily modified through conventional chemical, physical, and biochemical means. In addition, known pharmacological agents may be subject to directed or random chemical modifications, such as acylation, alkylation, esterification, amidification, etc.

The compound formulations may conveniently be presented in unit dosage form, e.g., tablets and sustained release capsules, and in liposomes, and may be prepared by any methods well know in the art of pharmacy. (See, for example, Remington: The Science and Practice of Pharmacy by Alfonso R. Gennaro (Ed.) 20th edition, Dec. 15, 2000, Lippincott, Williams & Wilkins; ISBN: 0683306472). Screening methods

Screening compounds for potential effectiveness of an adiponectin treatment in a subject with albuminuria can be accomplished by a variety of means well known by a person skilled in the art.

To screen the compounds described above for ability to target adiponectin or adiponectin signalling pathways, the test compounds are administered to the test system. In one embodiment the test system is a culture of podocyte cells or an assay for albuminuria. The podocyte cells can be a primary or an immortalized cell line. The podocytes may be obtained from an animal, including but not limited to, a fish such as zebrafish, a rodent such as a mouse or a rat, a rabbit, a non-human primate and a human. The screening for activity in cultured podocytes involves exposure of the cultures to test compounds, followed by measurement of albumin permeability across the monolayer, e.g., as described in the Examples herein. A change in permeability identifies a compound as a candidate for an agent that mimics or agonizes (or antagonizes) adiponectin activity. Alternatively, the test system can be an animal with albuminuria, including, but not limited to, a fish such as a zebrafish, a rodent such as a mouse or a rat, a rabbit, a non-human primate, and a human. In this instance, test compound is administered to the animal, and urine is monitored for albumin or protein content. A change in albuminuria identifies a compound as a candidate for an agent that mimics or agonizes (or antagonizes) adiponectin activity. As another alternative, the assay for albuminuria involves cells where the expression of adiponectin is reduced and/or knocked out. The screening for activity in such cells involves exposure of the cultures to test compounds, followed by measurement of adiponectin-mediated activities—a restoration of adiponectin-mediated activities identifies a test compound as a candidate adiponectin agonist. As another alternative, the system can be a transgenic mouse where adiponectin has been knocked out (see, e.g., Example 2). In this alternative, a reduction in albuminuria following exposure to a test compound identifies the test compound as a candidate adiponectin pathway agonist.

The test compounds can be administered, for example, by diluting the compounds into the medium wherein the cell is maintained, mixing the test compounds with the food where administered to an animal, or mixing liquid with cells from a biological sample, topically administering the compound in a pharmaceutically acceptable carrier to the animal, or by parenterally administering the compound. In some embodiments, the compounds are diluted into the media wherein the cell is maintained.

A variety of other reagents may also be included in the mixture. These include reagents such as salts, buffers, neutral proteins, e.g. albumin, detergents, etc. which may be used to facilitate optimal protein-protein and/or protein-nucleic acid binding and/or reduce non-specific or background interactions, etc. Also, reagents that otherwise improve the efficiency of the assay, such as protease inhibitors, nuclease inhibitors, antimicrobial agents, etc. may be used.

Screening for a compound that targets adiponectin or AMPK can be accomplished using measurements of cell growth or cell death. Screening may be accomplished by using measurements of adiponectin or AMPK transcription or translation. Screening may be accomplished by using measurements of adiponectin phosphorylation and/or AMPK-dependent phosphorylation. The abovementioned screening approaches may be used individually or in combination. To test targeting of adiponectin and/or AMPK by the test compound, a biological sample may be obtained from the test subject.

As noted above, screening assays are often carried out in vitro, for example, in cultured cells, in a biological sample, or fractions thereof. For ease of description, cell cultures, biological samples, and fractions are referred to as “samples” below. The sample is generally derived from an animal (e.g., any of the research animals mentioned above), preferably a mammal, and more preferably from a human.

Screening assays to detect adiponectin or AMPK transcription or expression are well known to the skilled artisan. Examples of such assays are described above in the section of the specification relating to diagnosis of adiponectin expression.

Kits

In another embodiment of the present invention, kits useful for the detection of Adiponectin expression are disclosed. Such kits may include any or all of the following: assay reagents, buffers, specific nucleic acids or antibodies (e.g. full-size monoclonal or polyclonal antibodies, single chain antibodies (e.g., scFv), or other gene product binding molecules), and other hybridization probes and/or primers, and/or substrates for polypeptide gene products.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

It is understood that the foregoing detailed description and the following examples are illustrative only and are not to be taken as limitations upon the scope of the invention. Various changes and modifications to the disclosed embodiments, which will be apparent to those skilled in the art, may be made without departing from the spirit and scope of the present invention. Further, all patents, patent applications and publications cited herein are incorporated herein by reference.

### EXAMPLES

A first series of experiments examined the functional relationships between adiponectin and albuminuria. These are discussed below.

Methods

Human Subjects: Subjects were recruited from an African American cohort that has been examined prospectively since late adolescence (23). Inclusion criteria were body mass index (BMI) greater than 30 and no known diabetes or kidney disease. Written informed consent was obtained from all subjects. Measurements included height, weight, calculated BMI, blood pressure, urine albumin/creatinine ratios assayed from timed overnight collections and the adipocytokines, adiponectin, IL6 and PAI-1. Urine albumin was measured by RIA (Diagnostic Products Corp., Los Angeles, Calif.). Plasma adiponectin was measured with an RIA kit from Linco, Inc. (St. Charies, Mo.). Plasma IL-6 and PAI-1 were measured by ELISA (R&D Systems, Minneapolis, Minn.). The protocol was approved by the Thomas Jefferson University Institutional Review Board.

Animals: Male AdKO mice on the C57BL16 genetic background, as previously described (24), were chosen for this study. All animal procedures were approved by the Institutional Animal Care and Use Committee of Thomas Jefferson University. Mice were given standard rodent chow (Purina 5010) and water ad lib. The genotype of each mouse was determined by PCR analysis of mouse tail DNA as previously described (24).

Adiponectin administration and animal studies: The mice were treated with 25 μg of recombinant human globular domain adiponectin (gAd; PepROTech, Rocky Hill, N.J.) subcutaneously twice daily for 10 consecutive days. The control animals were given PBS alone. 24-hour urines from each mouse were collected twice: one week before the injections and on the last day of the injections. The urine albumin and creatinine were measured with a mouse Albuwell ELISA kit and a Creatinine Companion kit (Exocell, Philadelphia, Pa.) (25,26). The mice were sacrificed five hours after the final gAd injection under anesthesia. Portions of kidney cortex were fixed in buffered formalin and embedded in paraffin. A portion of kidney cortical tissue was cut into 1 mm³ pieces and fixed in 2.5% glutaraldehyde

TABLE 2
Associations between selected variables and urine
Albumin/Creatinine

| | | | | Spearman Rank | |
|---|---|---|---|---|---|
| | Linear Regression | | | Sum Test | |
| Variable | R value | 95% C-I | P (2-tailed) | R value | P (2-tailed) |
| Adipo | −0.6392 | −0.8432 to −0.274 | 0.0024 | −0.5957 | 0.0056 |
| Age | −0.0279 | −0.4648 to 0.4198 | 0.9068 | −0.0641 | 0.9060 |
| BMI | 0.3129 | −0.1506 to 0.6636 | 0.1792 | 0.3895 | 0.0896 |
| SBP | 0.1616 | −0.3027 to 0.55639 | 0.4961 | 0.1659 | 0.4847 |
| DBP | 0.2444 | −0.2222 to 0.6200 | 0.2990 | 0.1414 | 0.5521 |
| T Chol | −0.0808 | −0.5054 to 0.3752 | 0.7348 | −0.2586 | 0.2709 |
| HDL | −0.4356 | −0.7363 to 0.0549 | 0.0549 | −0.5015 | 0.0243 |
| LDL | −0.0261 | −0.4633 to 0.4214 | 0.9131 | −0.1594 | 0.5021 |
| Trig | −0.2318 | −0.2349 to 0.6117 | 0.3254 | 0.0301 | 0.8998 |
| IL-6 | 0.0599 | −0.43932 to 0.4895 | 0.8020 | 0.3272 | 0.1591 |
| PAI-1 | 0.2301 | −0.2366 to 0.6105 | 0.3291 | 0.1023 | 0.6697 |

in Millonig solution and embedded in PolyBed 812 (Polysciences, Inc, Warrington, Pa.) for EM analysis.

Podocyte cell culture: Conditionally immortalized mouse podocytes were kindly provided by Dr. Peter Mundel (Albert Einstein College of Medicine, Bronx, N.Y.) and were cultured as previously described (27). The cells harbor a temperature-sensitive variant of the SV40 large T antigen (tsA58) that is inducible by γ-interferon and stable at 33° C. but rapidly degraded at 37° C. (27). At 33° C., the large T antigen allows for cellular proliferation. The proliferating cells are maintained in collagen I—coated flasks in RPMI-1640 media supplemented with 10% FBS and 10 units/ml mouse recombinant γ-interferon. When the cells have reached confluence, they are passaged and allowed to differentiate at 37° C. for 8-10 days without γ-interferon in Dulbecco's modified Eagle's medium containing 5.5 mmol/l glucose and 5% FCS.

For the permeability assay a modification of a previously published protocol (28) was adopted. Differentiated podocytes (0.2×10⁶ podocytes/well) plated on collagen coated 24 well Transwell plates (Corning) were serum-starved ovenight when confluent. Cells were then modulated with globular adiponectin (PepROTech Inc.) with or without 1 mM adenosine 9-p-D-arabinofuranoside (ARA-A) (Sigma, St. Louis, Mo.), for 24 h. Cells were then washed twice with PBS supplemented with 1 mM MgCl₂ and 1 mM CaCl₂. The upper compartment was refilled with 0.25 ml RPMI 1640 alone and the lower compartment with 0.5 ml RPMI 1640 supplemented with 40 mg/ml BSA and incubated for 2 hour at 37° C. Total protein concentration in the upper compartment was determined using a Bio-Rad protein assay (Bio-Rad Laboratories, Hercules, Calif.).

Western immunoblotting: Total protein from mouse podocytes was solubilized in lysis buffer containing 1% Triton X-100, Protease Inhibitor Cocktail (Mini Complete Protease Inhibitor Cocktail, Roche, Germany), PMSF, and phosphatase inhibitors. Protein was resolved on SDS-PAGE and transferred to a nitrocellulose membrane (Bio-Rad, Hercules, Calif.) as previously described (29). The membrane was immunoblotted initially with rabbit anti mouse phospho-AMPKα (p-AMPKα) monoclonal antibody or with rabbit anti AMPKα monoclonal (Cell Signaling Technology, New England Bio Labs, Boston, Mass.). Subsequently membranes were blotted with a secondary donkey anti-rabbit antibody (Amersham Biosciences, Piscataway, N.J.) conjugated to horseradish peroxidase (HRP). The HRP catalyzed chemiluminescence reaction was developed with SuperSignal West Pico substrate (Pierce Biotechnology Rockford, Ill.), allowing the detection of immunoreactive protein bands.

Immunohistochemistry: Immunocytochemistry was performed as described previously (30). Differentiated podocytes seeded on coverslips were serum-Starved overnight and globular adiponectin was added. After 24 hours of adiponectin introduction, cells were fixed in 3.7% paraformaldehyde. Incubation with primary antibody p-AMPKα at 1:50 dilution (Cell Signaling Technology) was performed in blocking solution at 4° C. overnight Cells were then incubated with secondary anti rabbit IgG conjugated to an immunofluorescent dye (Alexa 594, Invitrogen, Carisbad, Calif.) for 30 minutes at room temperature. Nuclear stain was performed with Hoechst 33342 (Invitrogen). Fluorescence pictures were obtained using confocal laser fluorescence microscope (LSM-510, Carl Zeiss, Jena, Germany). Immunostaining of paraffin-embedded mouse kidney was performed as described previously (26) with p-AMPKα (Thr172) rabbit monoclonal antibody (31). Quantitation of p-AMPKα positive cells was performed on 50 glomeruli from 4 mice in each group.

Statistical analysis: Data are summarized as arithmetic mean±SD or medians. Data that was normally distributed was used in a Pearson correlation analysis. Data that did not meet the criteria of normally distributed data was used in a Spearman Rank correlation. A value of p<0.05 was considered to indicate statistical significance. All reported values of P are 2-sided. Analyses were carried out using Graph Pad Prism software version 4.03 arid SPSS version 13.0 for the PC. Differences between data groups were evaluated for significance using independent t-test of data or 1-way ANOVA and Neuman-Keuls post-hoc tests.

### Example 1

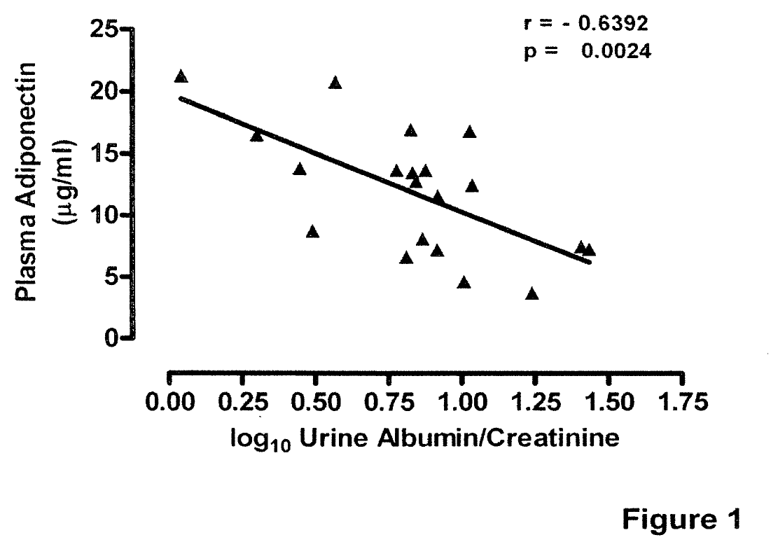

A total of 20 obese African American subjects who were on no medications for lipid or glycemic control were examined for relationships between albumin/creatinine ratios and adipocytokines. Characteristics of the subjects are presented in Table 1. Serum creatinine was normal (<1.4 mg/dl) and urine albumin/creatinine ratio was below 30 μg/mg in all subjects. In this sample of obese subjects, there was a statistically significant negative correlation between plasma adiponectin concentration and urinary albumin excretion, expressed as log transformed urine albumin:creatine ratio (r=−0.639, p<0.01) (FIG. 1). Of the other variables, there was a significant correlation between Albumin/creatinine ratios and HDL by the Spearman Rank Sum test (r=−0.502, p=0.024), but the correlation did not reach significance by Pearson linear regression (p=0.055). No significant correlations between urine albumin/creatinine ratios and plasma levels of IL-6 or PAI-1 were found. There were also no significant correlations between urine albumin/creatinine ratio and other clinical parameters including body mass index (BMI), blood pressure, total cholesterol, LDL and triglycerides.

TABLE 1
Characterisics of African American Subjects

| | Mean ± SD |
|---|---|
| Age (yr) | 40 ± 4.0 |
| M/F | 4/16 |
| Body mass index (BMI) | 40.14 ± 7.10 |
| SBP (mmHg) | 128 ± 13 |
| DBP (mmHg) | 73 ± 7 |
| Map (mmHg) | 91 ± 8 |
| Total Cholesterol (mg/dl) | 176 ± 47 |
| HDL (mg/dl) | 43 ± 10 |
| LDL (mg/dl) | 122 ± 43 |
| Triglycerides (mg/dl) | 102 ± 36 |
| U Alb/Creatinine (ug/mg) | 8.96 ± 7.01 (median = 7.18) |
| Plasma Adiponectin (ng/ml) | 12.0 ± 5.1 (median = 12.8) |
| Plasma IL-6 (pg/ml) | 7.0 ± 14 (median = 3.2) |
| Plasma PAI-1 (ng/ml) | 94.4 ± 37.1 (median = 99.4) |

TABLE 2
Association between selected variables and urine Albumin/Creatine.

| Variable | R value | 95% C-I | P (2-tailed) | R value | P (2-tailed) |
|---|---|---|---|---|---|
| Adipo | −0.6392 | −0.8432 to −0.274 | 0.0024 | −0.5957 | 0.0056 |
| Age | −0.0279 | −0.4648 to 0.4198 | 0.9068 | −0.0641 | 0.906 |
| BMI | 0.3129 | −0.1506 to 0.6636 | 0.1792 | 0.3895 | 0.0896 |
| SBP | 0.1616 | −0.3027 to 0.55639 | 0.4961 | 0.1659 | 0.4847 |
| DBP | 0.2444 | −0.2222 to 0.6200 | 0.299 | 0.1414 | 0.5521 |
| T Chol | −0.0808 | −0.5054 to 0.3752 | 0.7348 | −0.2586 | 0.2709 |
| HDL | −0.4356 | −0.7363 to 0.0549 | 0.0549 | −0.5015 | 0.0243 |
| LDL | −0.0261 | −0.4633 to 0.4212 | 0.9131 | −0.1594 | 0.5021 |
| Trig | −0.2318 | −0.2349 to 0.6117 | 0.3254 | 0.0301 | 0.8998 |
| IL-6 | 0.0599 | −0.43932 to 0.4895 | 0.802 | 0.3272 | 1591 |
| PAI-1 | 0.2301 | −0.2366 to 0.6105 | 0.3291 | 0.1023 | 0.6697 |

### Example 2

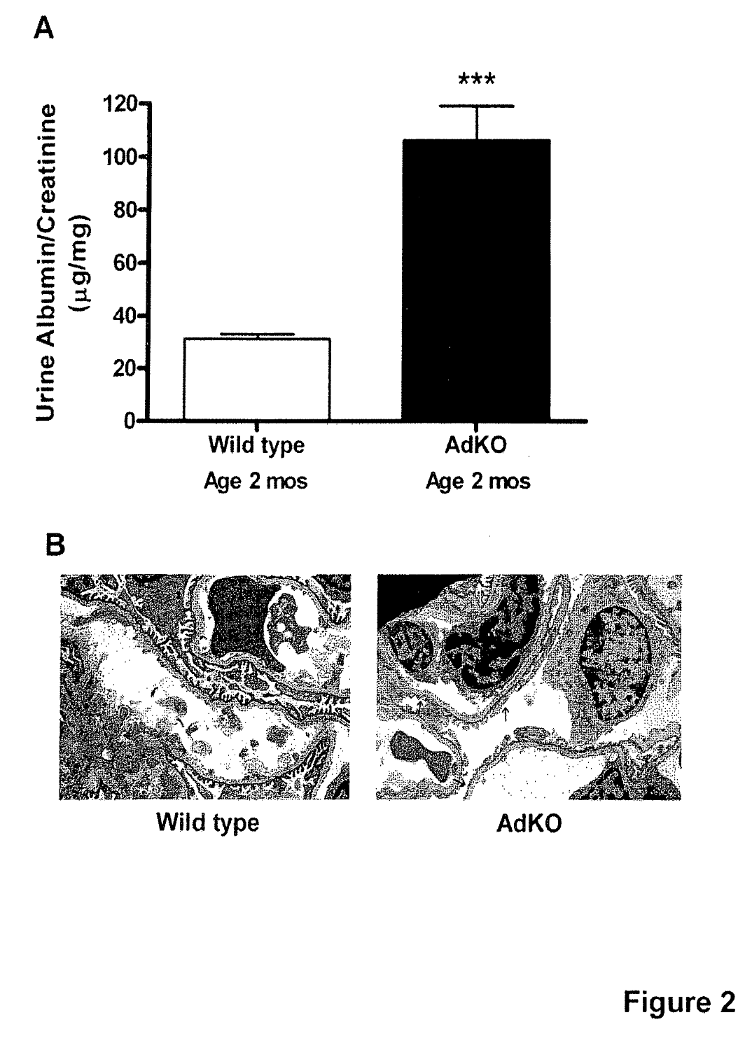

In order to determine whether adiponectin may be a causative factor for albuminuria we examined the adiponectin knock-out mouse (AdKO). This mouse has been reported to have normal glucose tolerance and insulin sensitivity when fed normal rodent chow (24). The AdKO mice had normal levels of blood pressure, lipids and body weight. The degree of albuminuria in the AdKO mice was increased by >2-fold between 1-3 months of age and increased further at 4 months of age (106.2±12.9 vs. 31.3±1.8 μg albumin/mg creatinine, p<0.001), compared to age and sex-matched wild type mice (FIG. 2A). To determine how adiponectin may contribute to albuminuria we examined electron microscopic sections of the glomeruli from wild type and AdKO mice. Podocyte foot processes were segmentally fused in the AdKO glomeruli (FIG. 2B). Glomerular basement membrane thickness, endothelial cells, and mesangial cells were similar in appearance to normal wild type mice.

### Example 3

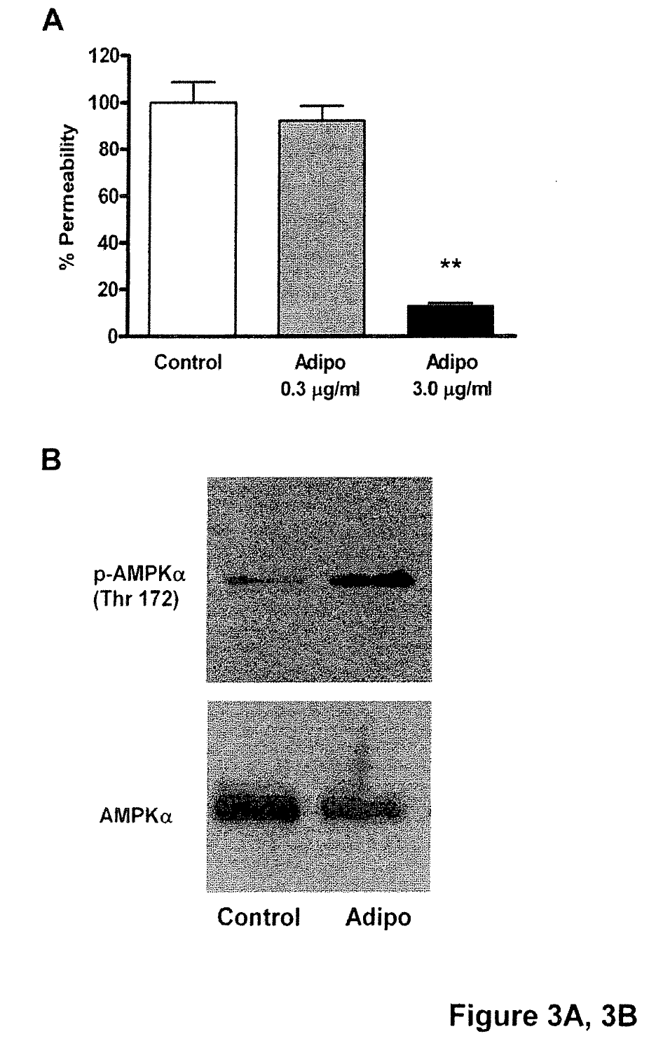

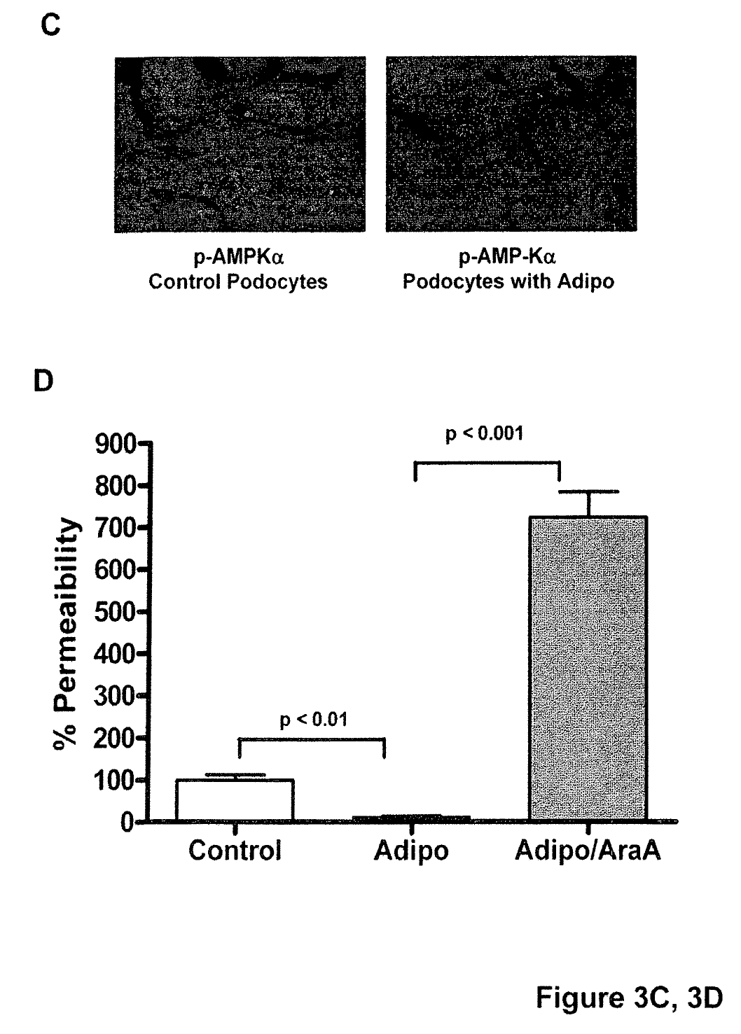

To determine if adiponectin had direct effects on podocyte function, a permeability assay was used to measure albumin permeability across a differentiated podocyte cell monolayer in vitro. In serum free conditions without adiponectin, there was evidence of transmonolayer permeability of albumin across podocytes cultured on porous membranes (FIG. 3A). Permeability was reduced with the addition of adiponectin (FIG. 3A), indicating that the integrity of the podocyte monolayer was improved.

Among the pathways implicated in adiponectin action, a major role for its action in liver and skeletal muscle as well as its protective effect in cardiomyocytes appears to involve the AMPK pathway (32). Podocytes in serum-free media had low AMPK activity, as evidenced by phosphorylation of the AMPK α subunit on Thr-172 (FIGS. 3B and 3C). AMPKα phosphorylation was increased with addition of adiponectin. A functional role for AMPK was demonstrated as inhibition of AMPK with a specific inhibitor (ARA-A) prevented the protective effects of adiponectin on podocyte permeability (FIG. 3D).

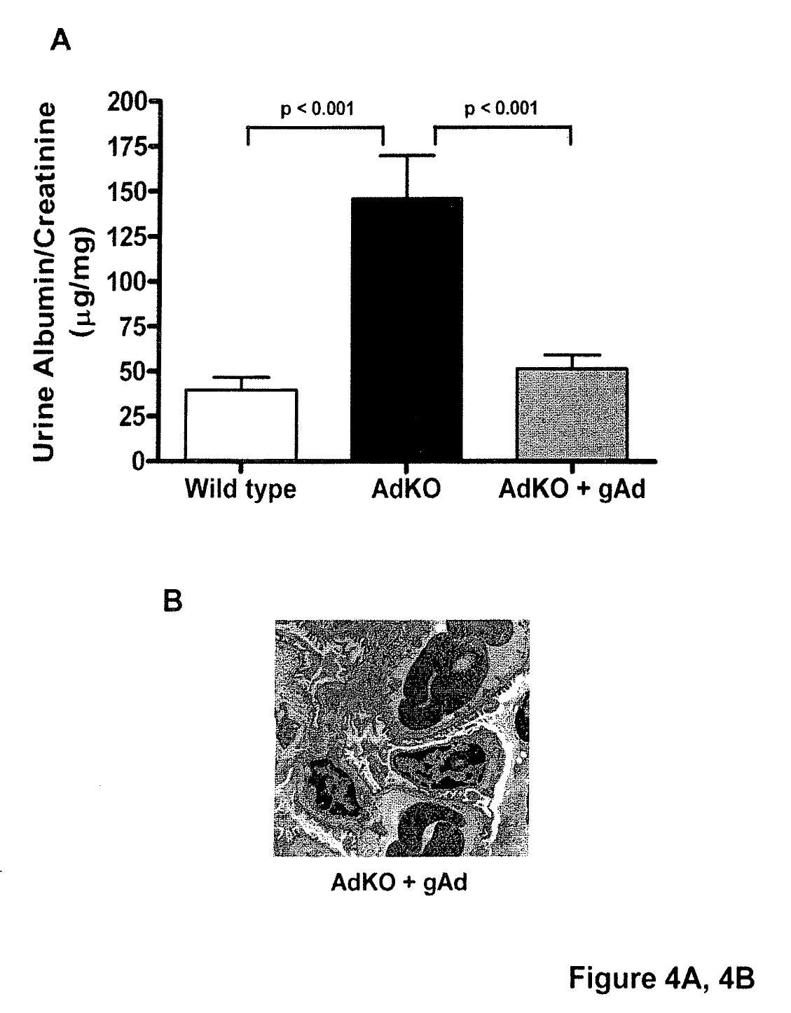

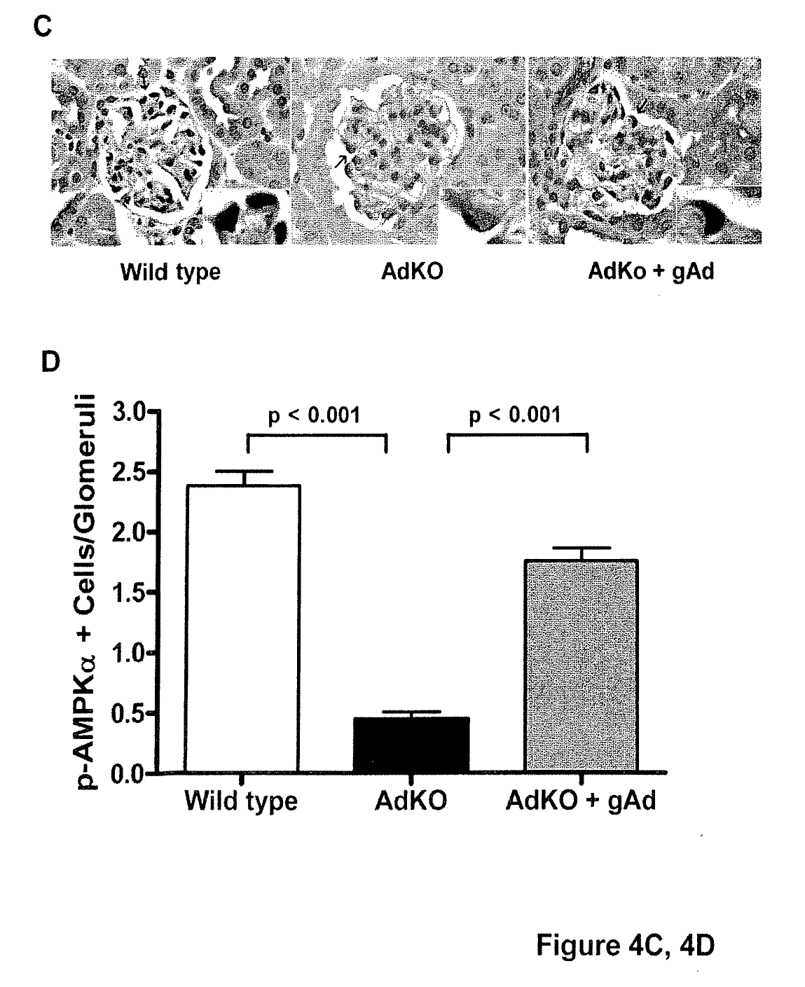

To determine if adiponectin replacement would be sufficient to restore normal permeability, the recombinant globular domain of adiponectin was administered to adiponectin deficient mice at 4 months of age, after the onset of increased albuminuria and foot process effacement. Adiponectin administration for 10 days normalized albuminuria levels in AdKO mice (FIG. 4A) and improved podocyte foot process effacement (FIGS. 4B). Reduced AMPK activity in the AdKO mouse glomerular podocytes (4C and 4D) was improved with adiponectin treatment.

A protective role of AMPK has been demonstrated in several cell types (35,36). The studies described herein demonstrate that AMPK plays a critical role in permeability of podocytes and that AMPK activity in podocytes is regulated by adiponectin. AMPK is a hetero-trimeric signaling kinase and a critical energy sensing pathway with important functions to stimulate glucose uptake. It has been demonstrated that the effect of adiponectin on various cell types involves AMPK as well as other pathways, such as the cAMP-PKA pathway (35). In podocytes, reduction in AMPK is associated with increased permeability, possibly due to effects on slit diaphragm proteins or on cytoskeleton. As the podocyte is the major cell type protecting the glomerulus from leaking albumin into the urinary space, specific podocyte proteins regulated by AMPK will be of major interest and likely to be involved in linking obesity, hypoadiponectinemia and albuminuria.

A second series of experiments expanded on the initial investigations and is described in the following Examples.

Albuminuria in the so-called high normal range (10-30 ug/mg) has been identified as a risk factor for cardiovascular disease (44). Renal dysfunction may contribute to overall CVD by also promoting vascular thickening and vascular calcification (7), as well as by activating inflammatory pathways (8). It has also been recognized that insulin resistance is closely associated with oxidant stress, early decline in renal function, and albuminuria (1, 9).

Adiponectin concentration in the blood ranges from 5-30 μg/ml in normal individuals and may exist in multiple forms including high molecular weight trimers and polymers. Adiponectin may also be cleaved into a collagenous and globular domain. The globular form of adiponectin may be derived from cleavage by neutrophil elastase and has been found in both human and mouse plasma (45). Adiponectin has largely beneficial effects, as it improves insulin sensitivity and decreases the adverse effects of inflammatory mediators in vascular cells (11, 12). Both the globular and full length forms of adiponectin have been found to bind to two adiponectin receptors (AdipoR1 and AdipoR2) (46) and signal via stimulation of 5′-AMP activated protein kinase (AMPK) as well as potentially other intracellular pathways (46). Protective effects of adiponectin may involve reduction of oxidant stress, possibly by inhibition of NADPH oxidases as our group has shown in endothelial cells (35) and in myocardial tissue (47). Plasma adiponectin levels are reduced with increasing visceral obesity and tightly correlated with insulin resistance and development of type 2 diabetes mellitus (13). Interestingly in patients with type 1 diabetes a SNP in the adiponectin promoter showed linkage with diabetic nephropathy (48) and low plasma adiponectin levels were predictive of the development of coronary artery calcification (14, 15) suggesting an important role for adiponectin in development of kidney and macrovascular disease with hyperglycemia. In the African American (AA) population, low plasma adiponectin levels have been reported in obese subjects and may be predictive of the development of type 2 diabetes (16, 17). Of note, both diabetes (24) and AA ethnicity are strong pre-disposing risk factors for progressive kidney disease (19). Although both adiponectin levels and albuminuria are associated with CVD and kidney dysfunction, studies linking adiponectin with albuminuria have been inconclusive (20-22). Importantly, a role for adiponectin in the development of albuminuria in its early stages has not been demonstrated.

In the following studies, which build upon those described above in Examples 1-3, it is shown that circulating adiponectin levels had a strong negative correlation with the degree of albuminuria in non-diabetic obese AA subjects. In the adiponectin knockout mouse (Ad−/−) urinary levels of albumin and hydrogen peroxide were increased, and podocyte foot process effacement was evident. In vitro, adiponectin potently decreased permeability to albumin through a monolayer of isolated cultured podocytes, largely via a AMPK-dependent pathway. In addition, adiponectin reduced the renal predominant NADPH oxidase (Nox4) (49) in podocytes. Treatment of Ad−/− mice with exogenous adiponectin decreased urine albumin and urinary hydrogen peroxide in association with a marked improvement in podocyte morphology, increased glomerular AMPK activity, and reduced glomerular Nox4. These data thus provide the first evidence that adiponectin contributes a protective role against albuminuria and that podocytes are a direct target of adiponectin action.

Methods

The following methods apply to the Examples that follow.

Animals: Male Ad−/− mice on the C57BL/6 genetic background were used as described in Example 2. Mice were given standard rodent chow (Purina 5010) and water ad lib and urine was collected in Nalgene metabolic cages at various time points. A cohort of wild type and Ad−/− mice A cohort of male wild type and Ad−/− mice at 2 months of age were made diabetic with a multiple low dose streptozotocin protocol as previously described (26). Blood glucose was measured with Accuchek. Urine collections in diabetic and non-diabetic mice were collected at baseline (2 months of age), 4 months (2 months of diabetes) and at 6 months (4 months of diabetes).

Interventional animal studies: The mice were treated with 25 μg of recombinant human globular domain adiponectin gAd (PepROTech, Rocky Hill, N.J.), administered 25 μg/mouse twice a day for 10 consecutive days or full-length adiponectin (fAd) was administered using an AAV vector (AAV2/8 CMV fAd virus 4×1011 GC/mouse for 10 days). A separate group of Ad−/− mice were treated with 5-aminoimidazole-4-carboxamide-1-beta-D-ribonucleoside (AICAR) with a single i.p. dose of 300 mg/kg. The control animals were given PBS alone or control AAV. 24-hour urines from each mouse were collected before treatment and on the last day of the treatment period. The urine albumin and creatinine were measured with a mouse Albuwell ELISA kit and a Creatinine Companion kit (Exocell, Philadelphia, Pa.) (26, 25). As an index of oxidant stress timed urine collections were also analyzed for hydrogen peroxide by Amplex red assay (Invitrogen/Molecular Probes, Carlsbad, Calif.) following the manufacturer's protocol. Portions of liver, muscle and kidney was snap-frozen in liquid nitrogen for RNA isolation. An additional aliquot of normal kidney was frozen in OCT for immunofluorescent staining. Portions of kidney cortex were fixed in buffered formalin and embedded in paraffin and a separate aliquot of kidney cortical tissue was cut into 1 mm3 pieces and fixed in 2.5% glutaraldehyde in Millonig solution and embedded in PolyBed 812 (Polysciences, Inc, Warrington, Pa.) for EM analysis.

Podocyte cell culture: Conditionally immortalized mouse podocytes were cultured as described in the Examples above.

Immunohistochemistry: Immunocytochemistry was performed as described previously (30). Differentiated podocytes seeded on coverslips were serum-starved overnight and globular adiponectin was added. After 24 hours of adiponectin introduction, cells were fixed in 3.7% paraformaldehyde. Incubation with primary antibody p-AMPK.a (Thr172) rabbit monoclonal antibody at 1:50 dilution (Cell Signaling Technology) was performed in blocking solution at 4° C. overnight. Cells were then incubated with secondary anti rabbit IgG conjugated to an immunofluorescent dye (Alexa 594, Invitrogen/Molecular Probes, Carlsbad, Calif.) for 30 minutes at room temperature. Nuclear stain was performed with Hoechst 33342 (Invitrogen/Molecular Probes). Fluorescence pictures were obtained using confocal laser fluorescence microscope (LSM-510, Carl Zeiss, Jena, Germany). Immunostaining of paraffin-embedded mouse kidneys was performed as described previously (26) with p-AMPK.a (Thr172) rabbit monoclonal antibody (31). Briefly, 4-μm thick paraffin sections were dewaxed, and antigen retrieval was performed by microwave for 15 minutes in antigen retrieval buffer (Citra Plus®, BioGenex, San Ramon, Calif.). To examine the expression of AMPK. and Nox4 in kidney tissues, primary antibodies were applied and incubated overnight (primary antibodies: pAMP-Ka (Thr172) rabbit monoclonal antibody at 1:50 dilution and Nox4 rabbit polyclonal antibody at 1:300 dilution). Then, biotin-labeled goat anti rabbit IgG (Invitrogen/Molecular Probes) at 1:150 dilution was applied as a secondary antibody. Endogenous peroxidase activity was blocked in 3% H2O2 in PBS, and avidin-biotin coupling reaction was performed on sections using Vectastain Elite Kit (Vector Laboratories, Burlingame, Calif.). As peroxidase substrate solution, DAB Substrate Kit, 3,3diaminobenzidine was used (Vector Laboratories). Quantitation of p-AMPKa positive cells was performed on 50 glomeruli from 4 mice in each group. For localization studies in mouse kidney tissue immunofluorescence with overlay was performed with the primary antibodies (rabbit monoclonal antibody p-AMPK.a (Thr172) at 1:50 dilution and polyclonal rabbit anti-Nox4 at 1:300 dilution) and double staining with the podocyte-specific mouse anti-synaptopodin antibody (Biodesign). Secondary antibodies for immunoflourescence and overlay was performed with Donkey anti-rabbit IgG (Alexa Fluor 594) at 1:150 dilution+anti-mouse IgG (Alexa Fluor 488) at 1:150 dilution. Images were captured by confocal laser fluorescence microscope.

RNA isolation and quantitative real time PCR analysis: Total RNA was isolated from liver, muscle, kidney, and differentiated podocytes using TRI-ZOL reagent, as previously described (52). Real time PCR was performed with cDNA from kidney cortex as previously described (52). The primers for mouse AdipoR1, AdipoR2, Nox1, Nox2, Nox4 and β-actin are as follows:

AdipoR1:
For;

(SEQ ID NO: 4)

GTT TGC CAC TCC CAA GCA C,
Rev;

(SEQ ID NO: 5)

GTA AAG TGC ATG GTG GGT AC,
Probe;

(SEQ ID NO: 6)

Fam AC CAC TCA AGC CAA GTC CCA GGA AC Tamra
AdipoR2:
For;

(SEQ ID NO: 7)

CCT GGC AAA TGT GAC ATC TG,
Rev;

(SEQ ID NO: 8)

CGT GGA AGT GAA CAA AGG CA,
Probe;

(SEQ ID NO: 9)

Fam CA CTC TCA TCA GCT CTT CCA CAT CTT TG Tamra
Nox1:
Forward;

(SEQ ID NO: 10)

CTT TTA TCG CTC CCA GCA GA,
Reverse;

(SEQ ID NO: 11)

CTC GCT TCC TCA TCT GCA AT,
Probe;

(SEQ ID NO: 12)

Fam CG TGA TTA CCA AGG TTG TCA TGA ACC CA Tamra
Nox2:
Forward;

(SEQ ID NO: 13)

TGC CAC CAG TCT GAA ACT CA,
Reverse;

(SEQ ID NO: 14)

CAG CAG GTC TGC AAA CCA CT,
Probe;

(SEQ ID NO: 15)

Fam AG GCA TGC GTG TCC CTG CAC AGC CA Tamra
Nox4:
Forward;

(SEQ ID NO: 16)

AGT AGT AGG AGA CTG GAC AG,
Reverse;

(SEQ ID NO: 17)

AAT GAA GGG CAG AAT CTC AGA,
Probe;

(SEQ ID NO: 18)

Fam TC CGG GAT TTG CTA CTG CCT CCA TCA AG Tamra
beta-actin:
Forward;

(SEQ ID NO: 19)

AAG AGC TAT AGA CTG CCT GA,
Reverse;

(SEQ ID NO: 20)

ACG GAT GTC AAC GTC ACA CT,
Probe;

(SEQ ID NO: 21)

Fam CA CTA TTG GCA ACG AGC GGT TCC G Tamra

Statistical analysis: Data are summarized as arithmetic means±SD or medians. Data that was normally distributed was used in a Pearson correlation analysis. Data that did not meet the criteria of normally distributed data was used in a Spearman Rank correlation. A value of p<0.05 was considered to indicate statistical significance. All reported values of P are 2-sided. Analyses were carried out using Graph Pad Prism software version 4.03 and SPSS version 13.0 for the PC. Differences between data groups were evaluated for significance using independent t-test of data or 1-way ANOVA and Neuman-Keuls post-hoc tests.

### Example 4

### Albuminuria and Oxidant Stress Increased in Ad−/− Mice

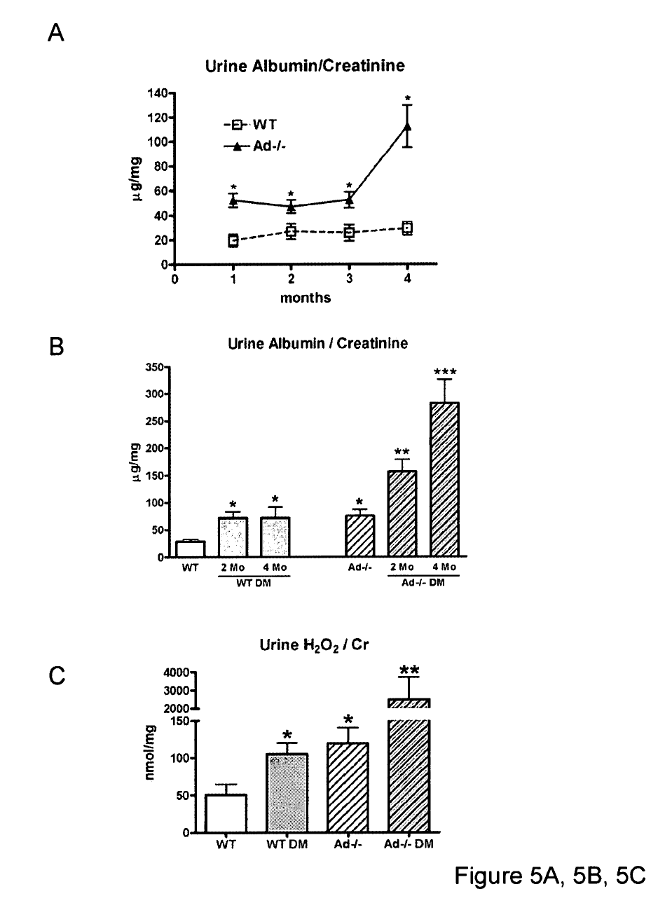

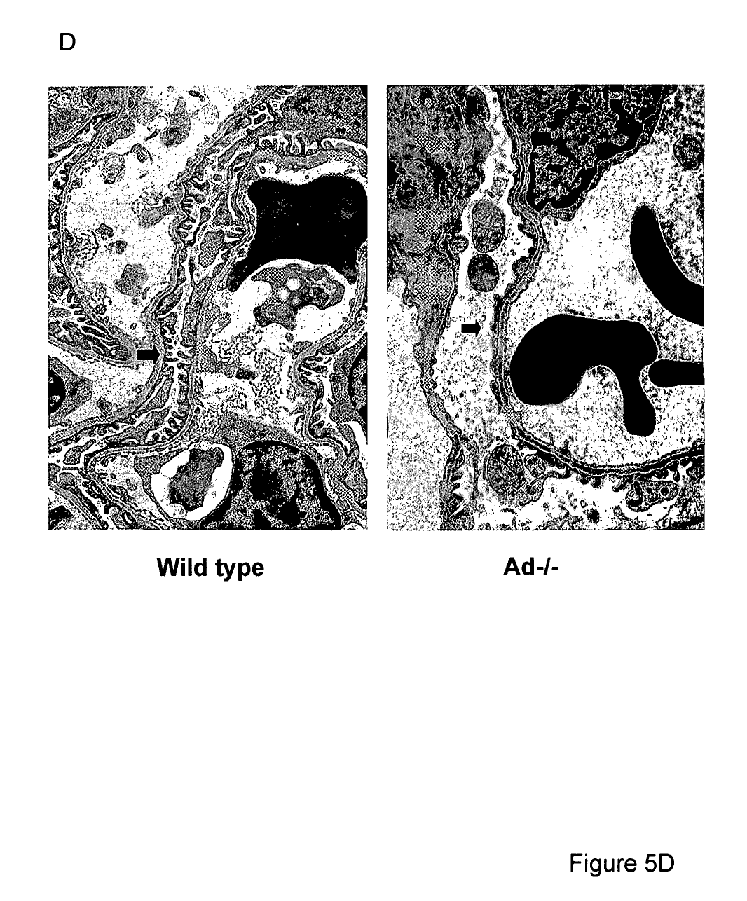

Wild-type and Ad−/− mice were also examined with diabetes. Induction of diabetes with multiple low dose streptozotocin in wild type C57B16 mice only modestly increased albuminuria, even with 4 months of diabetes (FIG. 5B). However, induction of type 1 diabetes in Ad−/− mice led to a significant increase in albuminuria within 2 months of diabetes and exhibited a progressive increase at 4 months of diabetes (FIG. 5B). Because oxidant stress is considered to be critical in the development of cardiovascular complications with states of adiponectin deficiency and because oxidant stress may be regulated by adiponectin (35, 47), urinary levels of hydrogen peroxide were measured. Urinary hydrogen peroxide was chosen as a measure of oxidant stress as it is relatively stable, at high concentrations in the urine, and reflects both systemic and renal oxidant stress (53, 54). Urinary hydrogen peroxide levels were increased in Ad−/− mice (FIG. 5C). With the additional stress of hyperglycemia and diabetes there was a marked increase in urinary levels of hydrogen peroxide (FIG. 5C). The degree of hyperglycemia and body weight was similar in wild type and Ad−/− diabetic mice at 4 months of diabetes (blood glucose: WT 495±128 mg/dl, Ad−/−522±135 mg/dl, body weight: WT 27.2±2.7 grams, Ad−/−27.3±2.3 grams).

### Example 5

### Direct Effects of Adiponectin on Podocytes

To examine how adiponectin may contribute to albuminuria electron microscopic sections of the glomeruli from wild type and Ad−/− mice were examined at 3 months of age. Podocyte foot processes were segmentally fused in the Ad−/− glomeruli (FIG. 5D). Glomerular basement membrane thickness, endothelial cells, and mesangial cells were similar in appearance to normal wild type mice. Thus, adiponectin deficiency is associated with podocyte dysfunction.

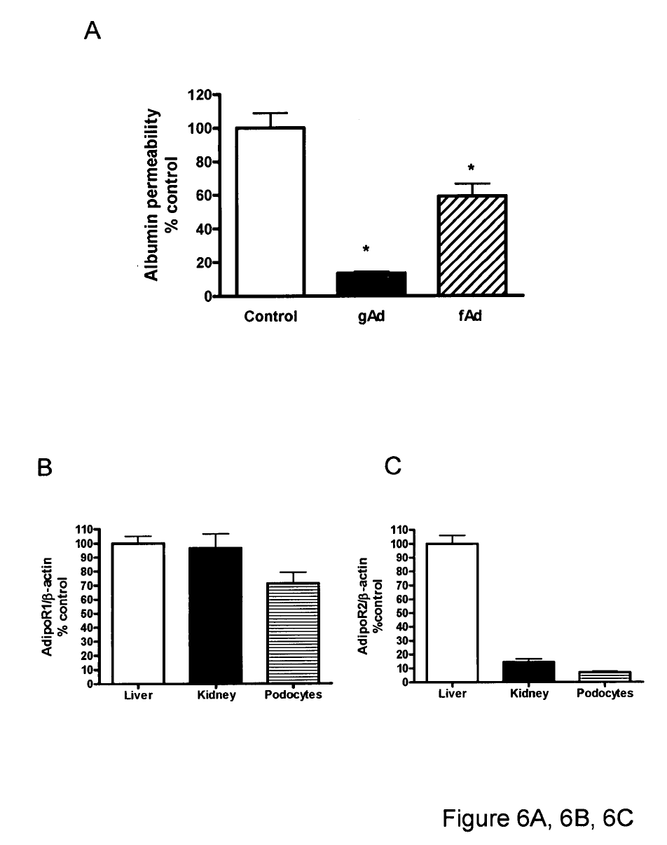

To further examine direct effects of adiponectin on podocyte function, a permeability assay as used in Example 3 was used to measure albumin permeability across a differentiated podocyte cell monolayer in vitro. As compared to the degree of permeability of albumin across podocytes cultured on porous membranes in serum free conditions without adiponectin, permeability was significantly reduced with the addition of globular or full length adiponectin (FIG. 6A). These data indicate a direct action of adiponectin on podocytes independent of the systemic/metabolic effects of adiponectin. These data also further demonstrate that the globular domain of adiponectin is sufficient for podocyte permeability-regulating activity of adiponectin.

By real time PCR, kidney and podocytes expressed similar amount of AdipoR1, but much less of AdipoR2, as compared to mouse liver tissue. This is in agreement with published data from Northern analysis of mouse liver and kidney for AdipoR1 and AdipoR2 (55).

### Example 6

### Adiponectin Stimulates AMPK in Podocytes

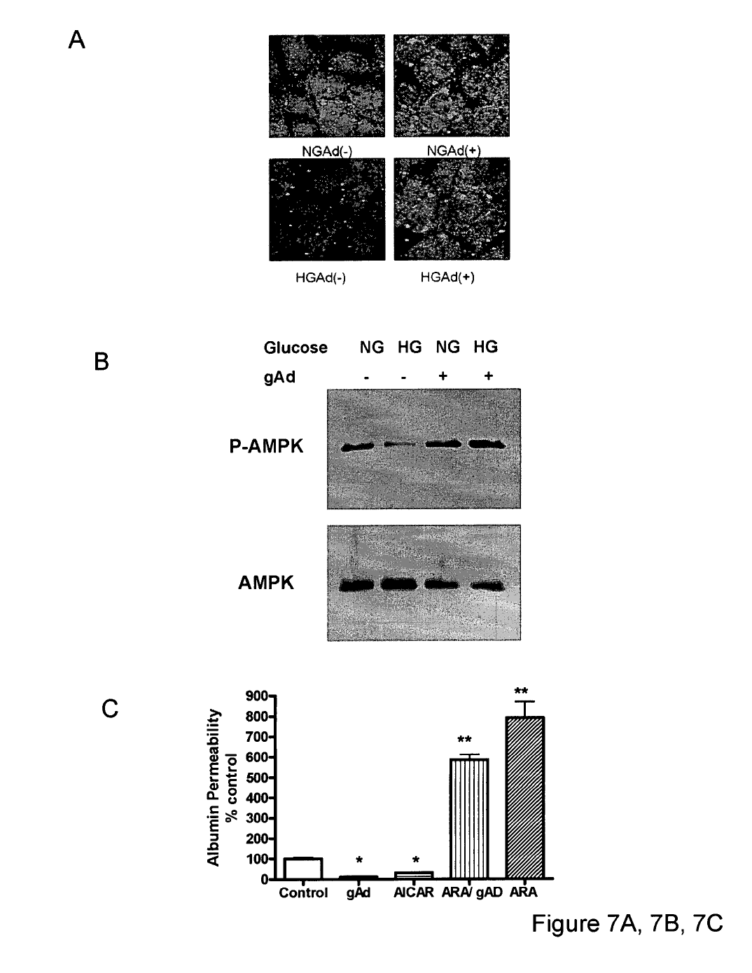

The involvement of AMPK in the effects of adiponectin was further evaluated using specific activation of AMPK in addition to specific inhibition of AMPK activity. The baseline AMPK activity in podocytes cultured in normal glucose was increased with addition of adiponectin (FIGS. 7A and 7B). AMPKa phosphorylation was further reduced by high glucose exposure, but prevented by exposure to adiponectin (FIG. 7A, 7B). A functional role for AMPK was demonstrated as a specific activator of AMPK (AICAR) had a similar effect as adiponectin to reduce permeability of podocytes to albumin (FIG. 7C). As discussed in Example 3, a specific inhibitor of AMPK (ARA-A) increased permeability to albumin either alone, or in the presence of adiponectin (FIG. 7C).

### Example 7

### Adiponectin Replacement Restores Normoalbuminuria

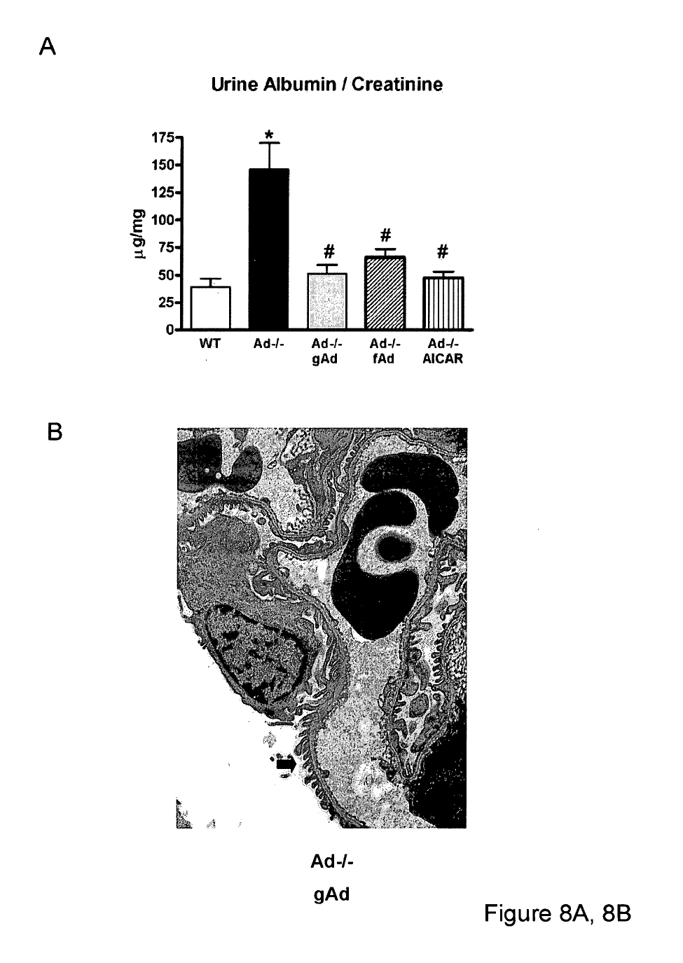

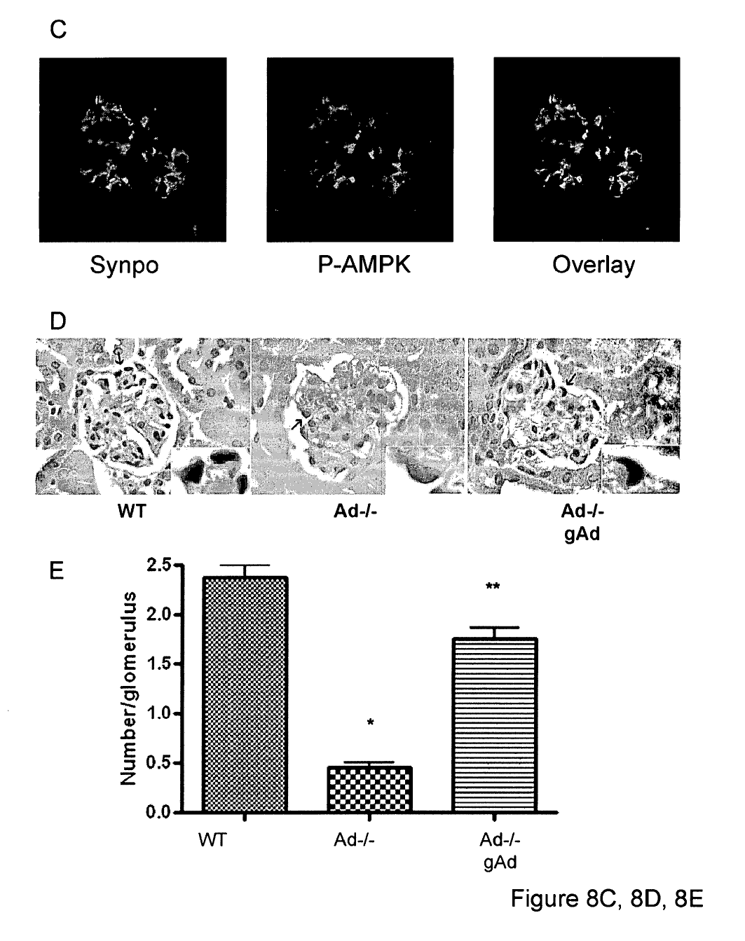

To determine if adiponectin replacement would be sufficient to prevent increased urinary levels of albumin, gAd or fAd was administered to Ad−/− mice at 4 months of age, after the onset of increased albuminuria and foot process effacement. Adiponectin administration normalized albuminuria in Ad−/− mice (FIG. 8A). A role for AMPK was also demonstrated, as AICAR administration restored albuminuria in Ad−/− mice (FIG. 8A). Adiponectin administration restored podocyte foot processes in Ad−/− mice (FIG. 8B). AMPK activity in glomeruli was measured with an antibody to phospho-AMPK and found to be present primarily in podocytes of glomeruli of wild type mice (FIG. 8C). AMPK activity was reduced in Ad−/− mouse glomerular cells and improved with adiponectin treatment (8D and 8E).

### Example 8

### Oxidant Stress Reduced by Adiponectin: Role of Nox4

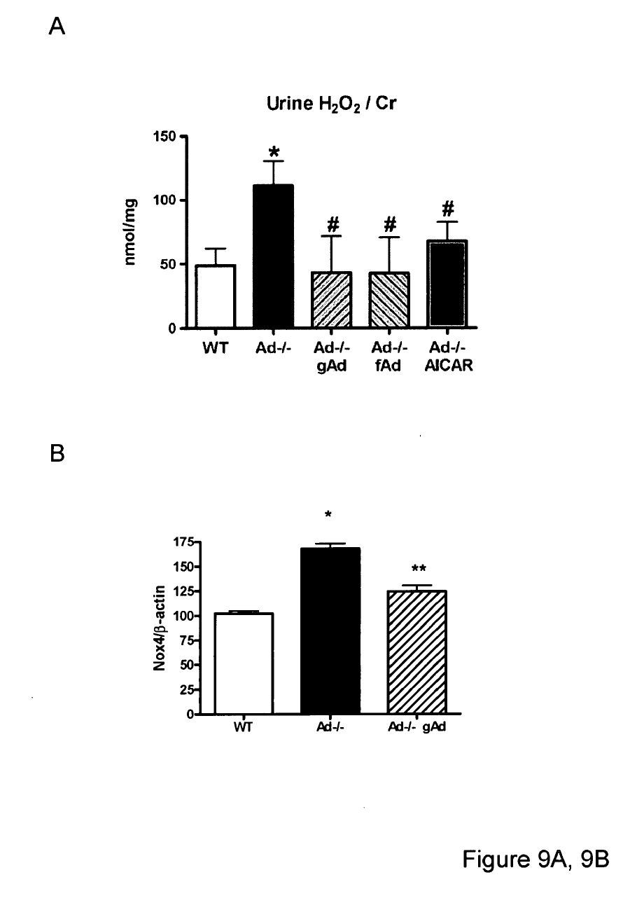

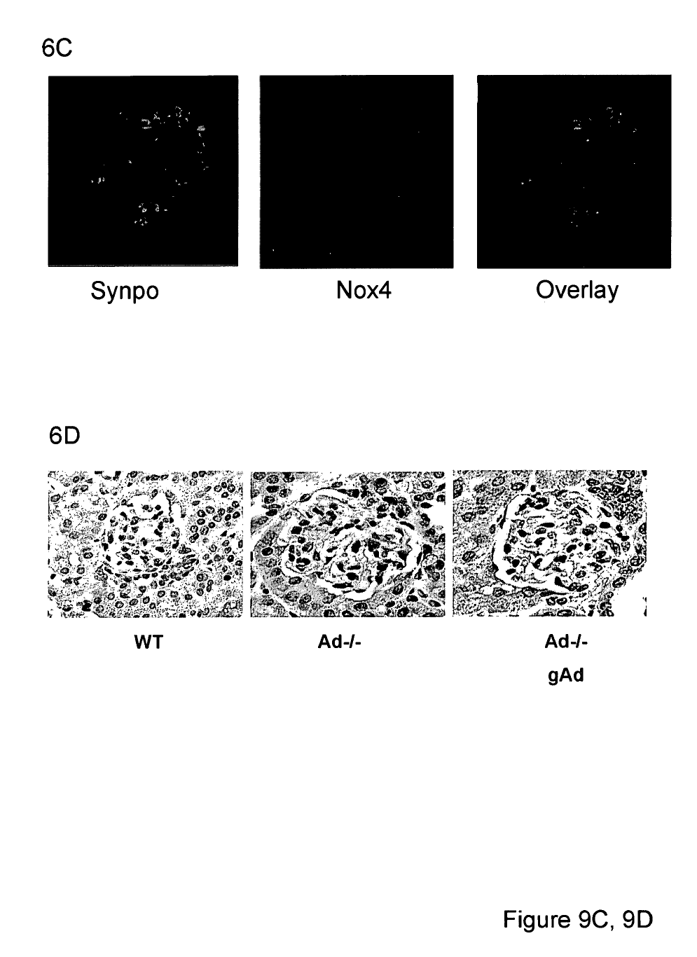

As oxidant stress has been linked to podocyte dysfunction and albuminuria (56), it was examined whether exogenous adiponectin regulated oxidant stress in the Ad−/− mice. The source of oxidant production was also evaluated. Increased urinary levels of hydrogen peroxide in 4 month-old Ad−/− mice was reduced with treatment with gAd, fAd or AICAR (FIG. 9A). Several NADPH oxidases have been described with Nox4 being most highly expressed in the kidney. By real time PCR Nox4 was significantly expressed and increased in renal Nox4 mRNA in Ad−/− kidneys and reduced to control levels with gAd treatment. Nox1 and Nox2 were expressed at low levels in the kidney and not increased in Ad−/− mice (data not shown). By immunofluorescence with double staining, glomerular Nox4 was clearly present in podocytes of WT kidneys (FIG. 9C) as well as in other glomerular and tubular cells. Glomerular Nox4 in Ad−/− kidneys was increased and reduced with gAd treatment (FIG. 9E).

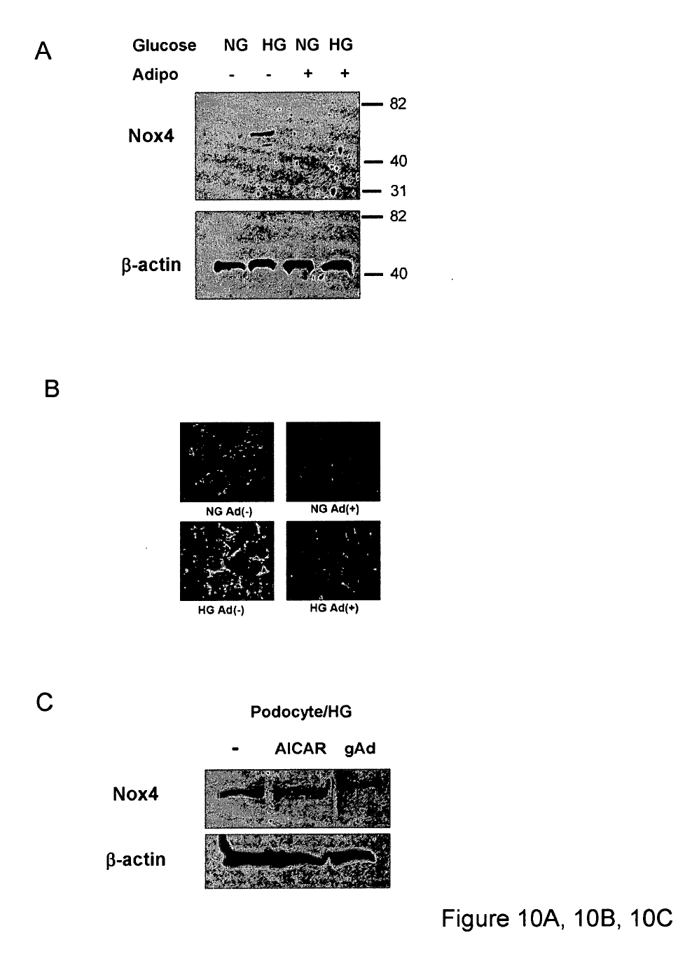

Nox4 protein was evident in podocyte cell culture grown in serum-free conditions and further increased with high glucose exposure (FIG. 10A). By confocal analysis podocyte Nox4 was primarily peri-nuclear and at cell periphery (FIG. 10B). Addition of adiponectin for 16 h was sufficient to suppress podocyte Nox4 (FIG. 10A, 10B). Furthermore, the AMPK activator AICAR reduced Nox4 protein levels to a similar degree as adiponectin (FIG. 10C).

These Examples demonstrate that Ad−/− mice have increased levels of albuminuria and urinary hydrogen peroxide, and havepodocyte dysfunction, indicating that adiponectin deficiency contributes to altered permeability, albuminuria, and oxidant stress. While not wishing to be bound by theory, the mechanism of podocyte dysfunction appears to be contributed via AMPK regulation by adiponectin, as podocyte permeability is improved with adiponectin treatment or AMPK activator and permeability is increased by AMPK inhibition. Additionally, the NADPH oxidase Nox4 is present in podocytes, regulated by adiponectin and may also contribute to podocyte dysfunction. Administration of adiponectin to the Ad−/− mice normalized albuminuria and oxidant stress, improved podocyte foot processes, increased glomerular AMPK activity, and reduced glomerular Nox4.

African Americans have a disproportionate and excessive representation of ESRD (33). African Americans also have high rates of obesity which heightens risk for kidney and cardiovascular disease. Low adiponectin levels have been identified in obese African Americans and are also associated with susceptibility to diabetes (16, 17). A relationship between low adiponectin levels and albuminuria in the micro- and overt range has been reported in populations with essential hypertension (21). The studies described herein link low adiponectin levels with albuminuria in the obese AA population. It is important to note that this correlation was noted in a population before the onset of diabetes and overt renal dysfunction. A similar observation was recently reported in patients with essential hypertension (21). The degree of albuminuria in the cohort examined herein was within the so-called normal range and thus represents a very early manifestation of kidney disease in association with obesity and insulin resistance. That low adiponectin levels are tightly correlated to this early rise in albuminuria is a primary conclusion supported by the clinical data.

Chronic kidney disease is a strong risk factor for cardiovascular disease mortality (1, 3-5). To some degree, the increased risk of mortality and CVD in chronic kidney disease may be explained by levels of adipokines, including elevated pro-inflammatory adipokines and reduction in adiponectin (8, 10). Adiponectin reduction has been well documented in states of obesity and prediabetes (34) and these conditions are often associated with microalbuminuria (9). However, it should be noted that subsequent to the development of overt proteinuria and renal insufficiency (20, 22) there is a reported increase in plasma adiponectin levels. However, several independent groups have reported that a region of chromosome 3q contains a susceptibility locus for diabetic nephropathy in patients with both type 1 (57, 58) and type 2 diabetes (59, 60) and one group evaluated 14 candidate genes on chromosome q and found the strongest linkage with a SNP for the promoter of adiponectin (48). The studies described in these Examples demonstrate a negative correlation with adiponectin and low levels of albuminuria in patients, demonstrate that adiponectin deficient mice have moderate increases in albuminuria, and that adiponectin deficiency dramatically increases the degree of albuminuria in diabetic mice. These findings clearly point to an important role for adiponectin in the initial development of increased albuminuria.

A protective role of AMPK has been demonstrated in several cell types (35, 36). The studies described herein demonstrate that AMPK plays a critical role in permeability of podocytes and that AMPK activity in podocytes is regulated by adiponectin. AMPK is a hetero-trimeric signaling kinase and a critical energy sensing pathway with important functions to stimulate glucose uptake. It has been demonstrated that the effect of adiponectin on various cell types involves AMPK as well as other pathways, such as the PPAR (55, 39) and cAMP-PKA pathway (35). As podocytes primarily express AdipoR1 it is likely that this receptor mediates adiponectin-induced AMPK activity. This postulate is consistent with recent data in AdipoR1 knockout mice (46). Functionally, these results demonstrate that reduction in AMPK activity is associated with increased permeability. In addition, a recent study using rat glomerular epithelial cells demonstrated that high glucose induced cell hypertrophy is regulated by AMPK (61) and may also contribute to podocyte dysfunction. As the podocyte is the major cell type protecting the glomerulus from leaking albumin into the urinary space, specific podocyte proteins regulated by AMPK will be of major interest and likely to be involved in linking obesity, hypoadiponectinemia and albuminuria.

One potential pathway by which adiponectin and AMPK activation may provide protection against albuminuria and podocyte permeability is via reduction of oxidant stress (35, 47, 62). Nox4 is a recently described non-phagocytic form of NAPDH oxidase that is highly expressed in the kidney (49). The studies described in these Examples demonstrate that podocytes express Nox4 and that adiponectin and AMPK regulate Nox4 protein in podocytes. Oxidant stress has been consistently linked with insulin resistance, obesity and adiponectin deficiency. The role of the kidney to contribute to oxidant stress has been largely ignored in settings of insulin resistance. The studies described in these Examples demonstrate that systemic adiponectin deficiency results in upregulation of Nox4 in the kidney and podocytes, and thus provides another critical link between obesity, insulin resistance and oxidant stress.

The results described herein indicate that several approaches can be used to lower the development of microalbuminuria and possibly CVD in populations at risk. Identification of low adiponectin levels and increased albuminuria canidentify a high risk profile with regard to kidney disease and CVD. In addition to the African American population, it is likely that similar findings will be made in other ethnic populations with obesity/insulin resistance (37) as well as type 1 diabetes (14, 15). Maneuvers to raise adiponectin levels, such as weight reduction, renin-angiotensin system blockade (38) and PPAR-gamma agonists (39), can be beneficial for reno-protection and for cardiovascular protection in at-risk populations. Treatment with metformin can be useful, as metformin raises AMPK activity independent of adiponectin (40). Inhibition of specific NADPH oxidase isoforms, such as Nox4, is expected to reduce podocyte dysfunction in states of adiponectin deficiency and has already been shown to benefit diabetic nephropathy in a rat model (63). Treatment with globular adiponectin or full length adiponectin is another option to treat podocyte dysfunction and albuminuria. It is contemplated that these approaches will be most successful in early stage kidney disease when podocyte function will be responsive to AMPK and before there is widespread podocyte depletion in situations of severe proteinuria.

In summary, circulating adiponectin levels are inversely related to albuminuria in obese African Americans without diabetes or overt kidney disease. Adiponectin plays a protective role to reduce albuminuria by directly affecting podocyte function via the AMPK pathway. These studies provide a strong pathobiologic rationale to intervene in the adiponectin-AMPK-Nox4 pathway to protect against albuminuria and treat or prevent early renal disease as well as associated cardiovascular disease.

### REFERENCES

The references cited herein and throughout the application are incorporated herein by reference.
1. Amann K, Wanner C, Ritz E. Cross-Talk between the Kidney and the Cardiovascular System. J Am Soc Nephrol 2006; 17:2112-9.

2. de Zeeuw D, Parving H-H, Henning R H. Microalbuminuria as an Early Marker for Cardiovascular Disease. J Am Soc Nephrol 2006:ASN.2006050517.

3. Go A, Chertow G, Fan D, McCulloch C, Hsu C. Chronic kidney disease and the risks of death, cardiovascular events, and hospitalization. N Engl J Med 2004; 351:1296-305.

4. Shlipak M G, Sarnak M J, Katz R, et al. Cystatin C and the Risk of Death and Cardiovascular Events among Elderly Persons. N Engl J Med 2005; 352(20):2049-60.

5. Anavekar N S, McMurray J J V, Velazquez E J, et al. Relation between Renal Dysfunction and Cardiovascular Outcomes after Myocardial Infarction. N Engl J Med 2004; 351(13):1285-95.

6. Ruggenenti P, Fassi A, Ilieva A P, et al. Preventing Microalbuminuria in Type 2 Diabetes. N Engl J Med 2004; 351(19):1941-51.

7. Davies M R, Lund R J, Mathew S, Hruska K A. Low turnover osteodystrophy and vascular calcification are amenable to skeletal anabolism in an animal model of chronic kidney disease and the metabolic syndrome. Journal of the American Society of Nephrology 2005; 16(4):917-28.

8. Becker B, Kronenberg F, Kielstein J T, et al. Renal Insulin Resistance Syndrome, Adiponectin and Cardiovascular Events in Patients with Kidney Disease: The Mild and Moderate Kidney Disease Study. J AM Soc Nephrol 2005; 16(4):1091-8.
9. Mykkanen L, Zaccaro D J, Wagenknecht L E, Robbins D C, Gabriel M, Haffner S M. Microalbuminuria is associated with insulin resistance in nondiabetic subjects: the insulin resistance atherosclerosis study. Diabetes 1998; 47(5):793-800.
10. Zoccali C, Mallamaci F, Tripepi G, et al. Adiponectin, Metabolic Risk Factors, and Cardiovascular Events among Patients with End-Stage Renal Disease. J AM Soc Nephrol 2002; 13(1):134-41.
11. Scherer P. Adipose Tissue: From Lipid Storage Compartment to Endocrine Organ. Diabetes 2006; 55:1537-45.
12. Goldstein B, Scalia, R. Adiponectin: a novel adipokine linking adipocytes and vascular function. J Clin Endocrinol Metab 2004; 89:2563-8.
13. Matsuzawa Y. Therapy Insight: adipocytokines in metabolic syndrome and related cardiovascular disease. Nat Clin Pract Cardiovasc Med 2006; 3:35-42.
14. Costacou T, Zgibor J C, Evans R W, et al. The prospective association between adiponectin and coronary artery disease among individuals with type 1 diabetes. The Pittsburgh Epidemiology of Diabetes Complications Study. Diabetologia 48(7): 1307-14 2005; 48(1):41-8.
15. Maahs D M, Ogden L G, Kinney G L, et al. Low plasma adiponectin levels predict progression of coronary artery calcification. Circulation 2005; 111(6):747-53.
16. Osei K, Gaillard T, Schuster D. Plasma adiponectin levels in high risk African-Americans with normal glucose tolerance, impaired glucose tolerance, and type 2 diabetes. Obes Res 2005; 13:179-85.
17. Bush N, Darnell B E, Oster R A et al. Adiponectin Is Lower Among African Americans and Is Independently Related to Insulin Sensitivity in Children and Adolescents. Diabetes 2005; 54:2772-8.
18. Ziyadeh F N, Sharma K. Overview: Combating Diabetic Nephropathy. J Am Soc Nephrol 2003; 14(5):1355-7.
19. System USRD. USRDS 2005 Annual Data Report: Atlas of End-Stage Renal Disease in the United States. Division of Kidney, Urologic, and Hematologic Diseases 2005; National Institute of Diabetic and Digestive Kidney Disease, NIH.
20. Schalkwijk C G, Chaturvedi N, Schram M T, Fuller J H, Stehouwer C D A, the EPCSG. Adiponectin Is Inversely Associated with Renal Function in Type 1 Diabetic Patients. J Clin Endocrinol Metab 2006; 91(1):129-35.
21. Tsioufis C, Dimitriadis K, Chatzis D, et al. Relation of Microalbuminuria to Andiponectin and Augmented C-Reactive Protein Levels in Men With Essential Hypertension. The American Journal of Cardiology 2005; 96(7):946.
22. Looker H C, Krakoff J, Funahashi T, et al. Adiponectin Concentrations Are Influenced by Renal Function and Diabetes Duration in Pima Indians with Type 2 Diabetes. J Clin Endocrinol Metab 2004; 89(8):4010-7.
23. Campbell K, Kushner, H., Falkner, B. Obesity and High Blood Pressure: A Clinical Phenotype for the Insulin Resistance Syndrome in African Americans. J Clin Hypertension 2004; 6:364-72.
24. Ma K, Cabrero A, Saha P K, et al. Increased beta-Oxidation but No Insulin Resistance or Glucose Intolerance in Mice Lacking Adiponectin. J Biol Chem 2002; 277(38):34658-61.
25. Ziyadeh F, Hoffman, B., Han, D., Iglesias-de la Cruz, C., Hong, S., Isono, M., Chen, S., McGowan, T., Sharma, K. Long-term prevention of renal insufficiency excess matrix gene expression and glomerular mesangial matrix expansion by treatment with monoclonal antitransforming growth factor-b antibody in db/db diabetic mice. Proc Natl Acad Sci USA 2000; 97:8015-20.
26. Susztak K, Bottinger E, Novetsky A, et al. Molecular Profiling of Diabetic Mouse Kidney Reveals Novel Genes Linked to Glomerular Disease. Diabetes 2004; 53(3):784-94.
27. Mundel P R J, Zuniga Mejia Borja A, Pavenstadt H, Davidson G R, Kriz W, Zeller R. Rearrangements of the cytoskeleton and cell contacts induce process formation during differentiation of conditionally immortalized mouse podocyte cell lines. Exp Cell Res 1997; 236:248-58.
28. Rico M, Mukherjee A, Konieczkowski M, et al. WT1-interacting protein and ZO-1 translocate into podocyte nuclei after puromycin aminonucleoside treatment AM J Physiol Renal Physiol 2005; 289(2):F431-41.
29. Sharma K, Deelman L, Madesh M, et al. Involvement of transforming growth factor-{beta} in regulation of calcium transients in diabetic vascular smooth muscle cells. Am J Physiol Renal Physiol 2003; 285(6):F1258-70.
30. McGowan T, Madesh M, Zhu Y, et al. TGF-b-induced Ca2+ influx involves the type III IP3 receptor and regulates actin cytoskeleton. AM J Physiol-Renal Fluid & Electrolyte Physiol 2002; 282:F910-F20.
31. Fraser S, Mount P, Hill R, et al. Regulation of the energy sensor AMP-activated protein kinase in the kidney by dietary salt intake and osmolality. Am J Physiol Renal Physiol 2005; 288(3):F578-86.
32. Ouchi N, Shibata R, Walsh K. Cardioprotection by adiponectin. Trends Cardiovasc Med 2006; 16:141-6.
33. Freedman B I, Bowden D W, Rich S S, et al. A genome scan for all-cause end-stage renal disease in African Americans. Nephrol Dial Transplant 2005; 20(4):712-8.
34. Kadowaki T, Yamauchi T, Kubota N, Hara K, Ueki K, To be K. Adiponectin and adiponectin receptors in insulin resistance, diabetes, and the metabolic syndrome. J Clin Invest 2006; 116(7):1784-92.
35. Ouedraogo R, Wu X, Xu S-Q, et al. Adiponectin Suppression of High-Glucose-Induced Reactive Oxygen Species in Vascular Endothelial Cells: Evidence for Involvement of a cAMP Signaling Pathway. Diabetes 2006; 55(6):1840-6.
36. Ouchi N, Kobayashi H, Kihara S, et al. Adiponectin Stimulates Angiogenesis by Promoting Cross-talk between AMP-activated Protein Kinase and Akt Signaling in Endothelial Cells. J Biol Chem 2004; 279(2):1304-9.
37. Retnakaran R, Anthony J. G. Hanley, and Bernard Zinrman. Does Hypoadiponectinemia Explain the Increased Risk of Diabetes and Cardiovascular Disease in South Asians? Diabetes Care 29 2006; 29:1950-4.
38. Yenicesu M, Yilmaz M I, Caglar K et al. Blockade of the Renin-Angiotensin System Increases Plasma Adiponectin Levels in Type-2 Diabetic Patients with Proteinuria. Nephron Clin Pract 2005; 99:c115-c21.
39. Yang B, Brown K K, Chen L, et al. Serum adiponectin as a biomarker for in vivo PPARgamma activation and PPARgamma agonist-induced efficacy on insulin sensitization/lipid lowering in rats. BMC Pharmacol 4(1):23 2004; 4(1):23.
40. Shaw R J, Lamia K A, Vasquez D, et al. The Kinase LKB1 Mediates Glucose Homeostasis in Liver and Therapeutic Effects of Metformin. Science 2005; 310(5754):1642-6.
41. Scherer, P. E., Williams, S., Fogliano, M., Baldini, G. & Lodish, H. F. A novel serum protein similar to C1q, produced exclusively in adipocytes. J Biol Chem 270, 26746-9 (1995).
42. Hu, E., Liang, P. & Spiegelman, B. M. AdipoQ is a novel adipose-specific gene dysregulated in obesity. J Biol Chem 271, 10697-703 (1996).
43. Maeda, K. et al. cDNA cloning and expression of a novel adipose specific collagen-like factor, apM1 (AdiPose Most abundant Gene transcript 1). Biochem Biophys Res Commun 221, 286 (1996).
44. Wachtell, K., Ibsen, H., Olsen, M., Borch-Johnsen, K., Lindholm, L., Mogensen, C., Dahlof, B., Devereux, R., Beevers, G., de Faire, U., et al. 2003. Albuminuria and cardiovascular risk in hypertensive patients with left ventricular hypertrophy: the LIFE study. Ann Int Med 139:901-906.
45. Fruebis, J., Tsao, T.-S., Javorschi, S., Ebbets-Reed, D., Erickson, M. R. S., Yen, F. T., Bihain, B. E., and Lodish, H. F. 2001. Proteolytic cleavage product of 30-kDa adipocyte complement-related protein increases fatty acid oxidation in muscle and causes weight loss in mice. PNAS 98:2005-2010.
46. Yamauchi, T., Nio, Y., Maki, T., Kobayashi, M., Takazawa, T., Iwabu, M., Okada-Iwabu, M., Kawamoto, S., Kubota, N., Kubota, T., et al. 2007. Targeted disruption of AdipoR1 and AdipoR2 causes abrogation of adiponectin binding and metabolic actions. Nat Med 13:332.
47. Tao, L., Gao, E., Jiao, X., Yuan, Y., Li, S., Christopher, T. A., Lopez, B. L., Koch, W., Chan, L., Goldstein, B. J., et al. 2007. Adiponectin Cardioprotection After Myocardial Ischemia/Reperfusion Involves the Reduction of Oxidative/Nitrative Stress. Circulation 115:1408-1416.
48. Vionnet, N., Tregouet, D., Kazeem, G., Gut, I., Groop, P.-H., Tarnow, L., Parving, H.-H., Hadjadj, S., Forsblom, C., Farrall, M., et al. 2006. Analysis of 14 Candidate Genes for Diabetic Nephropathy on Chromosome 3q in European Populations: Strongest Evidence for Association With a Variant in the Promoter Region of the Adiponectin Gene. Diabetes 55:3166-3174.
49. Geiszt, M., Kopp, J. B., Varnai, P., and Leto, T. L. 2000. Identification of Renox, an NAD(P)H oxidase in kidney. PNAS 97:8010-8014.
50. Mahadev, K., Motoshima, H., Wu, X., Ruddy, J., Arnold, R., Cheng, G., Lambeth, J., and Goldstein, B. 2004. The NAD(P)H oxidase homolog Nox4 modulates insulin-stimulated generation of H2O2 and plays an integral role in insulin signal transduction. Mol Cell Biol 24:1844-1854.
51. Hu, T., RamachandraRao, S. P., Siva, S., Valancius, C., Zhu, Y., Mahadev, K., Toh, I., Goldstein, B. J., Woolkalis, M., and Sharma, K. 2005. Reactive oxygen species production via NADPH oxidase mediates TGF-{beta}-induced cytoskeletal alterations in endothelial cells. Am J Physiol Renal Physiol 289:F816-825.
52. Zhu, Y., Casado, M., Vaulont, S., and Sharma, K. 2005. Role of Upstream Stimulatory Factors in Regulation of Renal Transforming Growth Factor-{beta} 1. Diabetes 54:1976-1984.
53. Djamali, A., Sadowski, E. A., Muehrer, R. J., Reese, S., Smavatkul, C., Vidyasagar, A., Fain, S. B., Lipscomb, R. C., Hullett, D. H., Samaniego-Picota, M., et al. 2007. BOLD-MRI assessment of intrarenal oxygenation and oxidative stress in patients with chronic kidney allograft dysfunction. Am J Physiol Renal Physiol 292:F513-522.
54. Zhang, L. F., Shigemoto*; Kosaka, Hiroaki. 2007. Effect of oestrogen on reactive oxygen species production in the aortas of ovariectomized Dahl salt-sensitive rats. J Hyper 25:407-414.
55. Yamauchi, T., Kamon, J., Ito, Y., Tsuchida, A., Yokomizo, T., Kita, S., Sugiyama, T., Miyagishi, M., Hara, K., Tsunoda, M., et al. 2003. Cloning of adiponectin receptors that mediate antidiabetic metabolic effects. [erratum appears in Nature. 2004 Oct. 28; 431(7012):1123]. Nature 423:762-769.
56. Susztak, K., Raff, A. C., Schiffer, M., and Bottinger, E. P. 2006. Glucose-Induced Reactive Oxygen Species Cause Apoptosis of Podocytes and Podocyte Depletion at the Onset of Diabetic Nephropathy. Diabetes 55:225-233.
57. Osterholm, A. M., He, B., Pitkaniemi, J., Albinsson, L., Berg, T., Sarti, C., Tuomilehto, J., and Tryggvason, K. 2006. Genome-wide scan for type 1 diabetic nephropathy in the Finnish population reveals suggestive linkage to a single locus on chromosome 3q. Kidney Int 71:140.
58. Moczulski, D. K., Rogus, J. J., Antonellis, A., Warram, J. H., and Krolewski, A. S.
1998. Major susceptibility locus for nephropathy in type 1 diabetes on chromosome 3q: results of novel discordant sib-pair analysis. Diabetes 47:11641169.
59. Imperatore, G., Hanson, R. L., Pettitt, D. J., Kobes, S., Bennett, P. H., and Knowler, W. C. 1998. Sib-pair linkage analysis for susceptibility genes for microvascular complications among Pima Indians with type 2 diabetes. Pima Diabetes Genes Group. Diabetes 47:821-830.
60. Bowden, D. W., Colicigno, C. J., Langefeld, C. D., Sale, M. M., Williams, A., Anderson, P. J., Rich, S. S., and Freedman, B. I. 2004. A genome scan for diabetic nephropathy in African Americans. Kidney Int 66:1517.
61. Lee, M.-J., Feliers, D., Mariappan, M. M., Sataranatarajan, K., Mahimainathan, L., Musi, N., Foretz, M., Viollet, B., Weinberg, J. M., Choudhury, G. G., et al. 2006. A role for AMP-activated protein kinase in diabetes-induced renal hypertrophy. Am J Physiol Renal Physiol: 00278.02006.
62. Alba, G., El Bekay, R., Alvarez-Maqueda, M., Chacon, P., Vega, A., Monteseirin, J., Santa Maria, C., Pintado, E., Bedoya, F. J., Bartrons, R., et al. 2004. Stimulators of AMP-activated protein kinase inhibit the respiratory burst in human neutrophils. FEBS Letters 573:219.
63. Gorin, Y., Block, K., Hernandez, J., Bhandari, B., Wagner, B., Barnes, J. L., and Abboud, H. E. 2005. Nox4 NAD(P)H Oxidase Mediates Hypertrophy and Fibronectin Expression in the Diabetic Kidney. J. Biol. Chem. 280:39616-39626.

TABLE 3
Wild-type Human adiponectin polypeptide sequence

(SEQ ID NO: 1)

| | |
|---|---|
| 1 | mlllgavlll lalpghdqet ttqgpgvllp lpkgactgwm |
| | agipghpghn gapgrdgrdg |
| 61 | tpgekgekgd pgligpkgdi getgvpgaeg prgfpgiqgr |
| | kgepgegayv yrsafsvgle |
| 121 | tyvtipnmpi rftkifynqq nhydgstgkf hcnipglyyf |
| | ayhitvymkd vkvslfkkdk |
| 181 | amlftydqyq ennvdqasgs vllhlevgdq vwlqvygege |
| | rnglyadndn dstftgflly |
| 241 | hdtn |

Wild-type Human globular domain:

(SEQ ID NO: 2)

| | |
|---|---|
| 111 | yrsafsvgle tyvtipnmpi rftkifynqq nhydgstgkf |
| | hcnipglyyf ayhitvymkd |
| 171 | vkvslfkkdk amlftydqyq ennvdqasgs vllhlevgdq |
| | vwlqvygege rnglyadndn |
| 231 | dstftgflly hd |

Wild-type Human adiponectin cDNA sequence

(SEQ ID NO: 3)

(details of intron/exon structure, etc. available
at NCBI GenBank Accession No. NM_004797)

| | |
|---|---|
| 1 | aggctgttga ggctgggcca tctcctcctc acttccattc |
| | tgactgcagt ctgtggttct |
| 61 | gattccatac cagaggggct caggatgctg ttgctgggag |
| | ctgttctact gctattagct |
| 121 | ctgcccggtc atgaccagga aaccacgact caagggcccg |
| | gagtcctgct tcccctgccc |
| 181 | aagggggcct gcacaggttg gatggcgggc atcccagggc |
| | atccgggcca taatggggcc |
| 241 | ccaggccgtg atggcagaga tggcacccct ggtgagaagg |
| | gtgagaaagg agatccaggt |
| 301 | cttattggtc ctaagggaga catcggtgaa accggagtac |
| | ccggggctga aggtccccga |
| 361 | ggctttccgg gaatccaagg caggaaagga gaacctggag |
| | aaggtgccta tgtataccgc |
| 421 | tcagcattca gtgtgggatt ggagacttac gttactatcc |
| | ccaacatgcc cattcgcttt |
| 481 | accaagatct tctacaatca gcaaaaccac tatgatggct |
| | ccactggtaa attccactgc |
| 541 | aacattcctg ggctgtacta ctttgcctac cacatcacag |
| | tctatatgaa ggatgtgaag |
| 601 | gtcagcctct tcaagaagga caaggctatg ctcttcacct |
| | atgatcagta ccaggaaaat |
| 661 | aatgtggacc aggcctccgg ctctgtgctc ctgcatctgg |
| | aggtgggcga ccaagtctgg |
| 721 | ctccaggtgt atggggaagg agagcgtaat ggactctatg |
| | ctgataatga caatgactcc |
| 781 | accttcacag gctttcttct ctaccatgac accaactgat |
| | caccactaac tcagagcctc |
| 841 | ctccaggcca aacagcccca aagtcaatta aaggctttca |
| | gtacggttag gaagttgatt |
| 901 | attatttagt tggaggcctt tagatattat tcattcattt |
| | actcattcat ttattcattc |
| 961 | attcatcaag taactttaaa aaaatcatat gctatgttcc |
| | cagtcctggg gagcttcaca |
| 1021 | aacatgacca gataactgac tagaaagaag tagttgacag |
| | tgctattttg tgcccactgt |
| 1081 | ctctcctgat gctcatatca atcctataag gcacagggaa |
| | caagcattct cctgttttta |
| 1141 | cagattgtat cctgaggctg agagagttaa gtgaatgtct |
| | aaggtcacac agtattaagt |
| 1201 | gacagtgcta gaaatcaaac ccagagctgt ggactttgtt |
| | cactagactg tgccctttta |
| 1261 | tagaggtaca tgttctcttt ggagtgttgg taggtgtctg |
| | tttcccacct cacctgagag |
| 1321 | ccattgaatt tgccttcctc atgaattaaa acctccccca |
| | agcagagctt cctcagagaa |
| 1381 | agtggttcta tgatgaagtc ctgtcttgga aggactacta |
| | ctcaatggcc cctgcactac |
| 1441 | tctacttcct cttacctatg tcccttctca tgcctttccc |
| | tccaacgggg aaagccaact |
| 1501 | ccatctctaa gtgctgaact catccctgtt cctcaaggcc |
| | acctggccag gagcttctct |
| 1561 | gatgtgatat ccactttttt tttttttgag atggagtctc |
| | actctgtcac ccaggctgga |
| 1621 | gtacagtgac acgacctcgg ctcactgcag cctccttctc |
| | ctgggtccaa gcaattattg |
| 1681 | tgcctcagcc tcccgagtag ctgagacttc aggtgcattc |
| | caccacacat ggctaatttt |
| 1741 | tgtattttta gtagaaatgg ggtttcgtca tgttggccag |
| | gctggtctcg aactcctggc |
| 1801 | ctaggtgatc cacccgcctc gacctcccaa agtgctggga |
| | ttacaggcat gagccaccat |
| 1861 | gcccagtcga tatctcactt tttattttgc catggatgag |
| | agtcctgggt gtgaggaaca |
| 1921 | cctcccacca ggctagaggc aactgcccag gaaggactgt |
| | gcttccgtca cctctaaatc |
| 1981 | ccttgcagat ccttgataaa tgcctcatga agaccaatct |
| | cttgaatccc atatctaccc |
| 2041 | agaattaact ccattccagt ctctgcatgt aatcagtttt |
| | atccacagaa acattttcat |
| 2101 | tttaggaaat ccctggtttt aagtatcaat ccttgttcag |
| | ctggacaata tgaatctttt |
| 2161 | ccactgaagt tagggatgac tgtgattttc agaacacgtc |
| | cagaattttt catcaagaag |
| 2221 | gtagcttgag cctgaaatgc aaaacccatg gaggaattct |
| | gaagccattg tctccttgag |
| 2281 | taccaacagg gtcagggaag actgggcctc ctgaatttat |
| | tattgttctt taagaattac |
| 2341 | aggttgaggt agttgatggt ggtaaacatt ctctcaggag |
| | acaataactc cagtgatgtt |
| 2401 | cttcaaagat tttagcaaaa acagagtaaa tagcattctc |
| | tatcaatata taaatttaaa |
| 2461 | aaactatctt tttgcttaca gttttaaatt ctgaacaatt |
| | ctctcttata tgtgtattgc |
| 2521 | taatcattaa ggtattattt tttccacata taaagctttg |
| | tctttttgtt gttgttgttg |
| 2581 | tttttaagat ggagtttccc tctgttgcca ggctagagtg. |
| | cagtggcatg atctcggctt |
| 2641 | actgcaacct ttgcctccca ggttcaagcg attcttctgc |
| | ctcagcctcc cgagtagctg |
| 2701 | ggaccacagg tgcctaccac catgccaggc taatttttgt |
| | atttttagta aagacagggt |
| 2761 | ttcaccatat tggccaggct ggtctcgaac tcctgacctt |
| | gtgatctgcc cgcctccatt |
| 2821 | tttgttgtta ttttttgaga aagatagata tgaggtttag |
| | agagggatga agaggtgaga |
| 2881 | gtaagccttg tgttagtcag aactctgtgt tgtgaatgtc |
| | attcacaaca gaaaacccaa |
| 2941 | aatattatgc aaactactgt aagcaagaaa aataaaggaa |
| | aaatggaaac atttattcct |
| 3001 | ttgcataata gaaattacca gagttgttct gtctttagat |
| | aaggtttgaa ccaaagctca |
| 3061 | aaacaatcaa gacccttttc tgtatgtcct tctgttctgc |
| | cttccgcagt gtaggcttta |
| 3121 | ccctcaggtg ctacacagta tagttctagg gtttccctcc |
| | cgatatcaaa aagactgtgg |
| 3181 | cctgcccagc tctcgtatcc ccaagccaca ccatctggct |
| | aaatggacat catgttttct |
| 3241 | ggtgatgccc aaagaggaga gaggaagctc tctttcccag |
| | atgccccagc aagtgtaacc |
| 3301 | ttgcatctca ttgctctggc tgagttgtgt gcctgtttct |
| | gaccaatcac tgagtcagga |
| 3361 | ggatgaaata ttcatattga cttaattgca gcttaagtta |
| | ggggtatgta gaggtatttt |
| 3421 | ccctaaagca aaattgggac actgttatca gaaataggag |
| | agtggatgat agatgcaaaa |
| 3481 | taatacctgt ccacaacaaa ctcttaatgc tgtgtttgag |
| | ctttcatgag tttcccagag |
| 3541 | agacatagct ggaaaattcc tattgatttt ctctaaaatt |
| | tcaacaagta gctaaagtct |
| 3601 | ggctatgctc acagtctcac atctggttgg ggtgggctcc |
| | ttacagaaca cgctttcaca |
| 3661 | gttaccctaa actctctggg gcagggttat tcctttgtgg |
| | aaccagaggc acagagagag |
| 3721 | tcaactgagg ccaaaagagg cctgagagaa actgaggtca |
| | agatttcagg attaatggtc |
| 3781 | ctgtgatgct ttgaagtaca attgtggatt tgtccaattc |
| | tctttagttc tgtcagcttt |
| 3841 | tgcttcatat attttagcgc tctattatta gatatataca |
| | tgtttagtat tatgtcttat |
| 3901 | tggtgcattt actctcttat cattatgtaa tgtccttctt |
| | tatctgtgat aattttctgt |
| 3961 | gttctgaagt ctactttgtc taaaaataac atacgcactc |
| | aacttccttt tctttcttcc |
| 4021 | ttcctttctt tcttccttcc tttctttctc tctctctctc |
| | tttccttcct tccttcctcc |
| 4081 | ttttctttct ctctctctct ctctctcttt ttttgacaga |
| | ctctcgttct gtggccctgg |
| 4141 | ctggagttca gtggtgtgat cttggctcac tgctacctct |
| | accatgagca attctcctgc |
| 4201 | ctcagcctcc caagtagctg gaactacagg ctcatgccac |
| | tgcgcccagc taatttttgt |
| 4261 | atttttcgta gagacggggt ttcaccacat tcgtcaggtt |
| | ggtttcaaac tcctgacttt |
| 4321 | gtgatccacc cgcctcggcc tcccaaagtg ctgggattac |
| | aggcatgagc catcacacct |
| 4381 | ggtcaacttt cttttgatta gtgtttttgt ggtatatctt |
| | tttccatcat gttactttaa |
| 4441 | atatatctat attattgtat ttaaaatgtg tttcttacag |
| | actgcatgta gttgggtata |
| 4501 | atttttatcc agtctaaaaa tatctgtctt ttaattggtg |
| | tttagacaat ttatatttaa |
| 4561 | taaaattgtt gaatttaaaa aaaaaaaaaa aa |

1. A method for decreasing the risk of developing, or reducing the effects of a kidney disorder in a subject, the method comprising administering to the subject an effective amount of a pharmaceutical composition comprising an adiponectin polypeptide and a pharmaceutically acceptable carrier.

2. The method of claim 1, further comprising, prior to said administering step, the step of measuring a level of adiponectin or RNA encoding adiponectin in a biological sample obtained from said subject and comparing said level to a baseline level.

3. The method of claim 1, wherein the kidney disorder is albuminuria.

4. The method of claim 1, wherein administration is prior to the onset of the kidney disorder.

5. The method of claim 1, wherein administration is post onset of the kidney disorder.

6. The method of claim 1, wherein administration is substantially concurrent with the kidney disorder.

7. The method of claim 1, wherein administration is within 24 hours after the onset of the kidney disorder.

8. The method of claim 1, wherein the adiponectin polypeptide is a trimer.

9. The method of claim 1, wherein the adiponectin polypeptide is the globular domain of adiponectin.

10. The method of claim 1, wherein the adiponectin polypeptide comprises a human adiponectin polypeptide.

11. The method of claim 9 wherein said globular domain comprises the sequence of SEQ ID NO: 2.

12. The method of claim 10 wherein said adiponectin polypeptide comprises the sequence of SEQ ID NO: 1.

13. The method of claim 1, wherein the subject is suffering from a condition selected from the group consisting of: hypertension; obesity; and kidney disease.

14. A method for decreasing the risk of developing, or reducing the effects of a kidney disorder in a subject, the method comprising administering to the subject an effective amount of a pharmaceutical composition comprising a nucleic acid construct comprising sequence encoding an adiponectin polypeptide operatively linked to control sequences sufficient for the expression of said adiponectin polypeptide.

15. The method of claim 14, further comprising, prior to said administering step, measuring a level of adiponectin or RNA encoding adiponectin in a biological sample obtained from said subject and comparing said level to a baseline level.

16. The method of claim 14, wherein said control sequences are inducible.

17. The method of claim 14 wherein said nucleic acid construct is comprised by a viral vector.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. A method for treating albuminuria, the method comprising administering to a subject in need thereof a pharmaceutical composition comprising an adiponectin polypeptide, in a pharmaceutically acceptable carrier.

30. (canceled)

31. A method for treating albuminuria, the method comprising administering to a subject in need thereof an inhibitor of Nox4 activity.

32. A method for treating or reducing the risk of developing a kidney disorder, the method comprising administering to a subject in need thereof an effective amount of a pharmaceutical composition comprising an AMPK agonist in a pharmaceutically acceptable carrier.

33. The method of claim 31 wherein said AMPK agonist comprises AICAR.

34. A method of identifying a subject having increased likelihood of having or developing a kidney disorder, the method comprising measuring a level of adiponectin or RNA encoding adiponectin in a biological sample obtained from a subject and comparing said level to a baseline level, wherein when the measured level of adiponectin or RNA encoding adiponectin is below said baseline level, the subject is identified as having an increased likelihood of having or being at risk of developing a kidney disorder.

35. (canceled)

36. (canceled)

37. The method of claim 34, wherein the biological sample is selected from the group consisting of serum, whole blood, plasma, urine, and a tissue sample.

38. The method of claim 34, wherein the biological sample is a urine sample.

39. (canceled)

40. (canceled)

41. (canceled)

42. (canceled)

43. A kit for treating a subject suffering from a kidney disorder, the kit comprising a nucleic acid containing a segment encoding an adiponectin polypeptide, operatively linked to control sequences sufficient for the expression of said adiponectin polypeptide, or a pharmaceutical composition comprising an adiponectin polypeptide, and a pharmaceutically aceptable carrier or excipient.

44. The kit of claim 43 wherein said nucleic acid is comprised by a viral vector.


SEQUENCE LISTING (21 sequences)

SEQ ID NO: 1 - protein, 244 aa, Homo sapiens
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 2 - protein, 132 aa, Homo sapiens
YRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHD

SEQ ID NO: 3 - DNA, 4592 bp, Homo sapiens
aggctgttgaggctgggccatctcctcctcacttccattctgactgcagtctgtggttctgattccataccagaggggctcaggatgctgttgctgggagctgttctactgctattagctctgcccggtcatgaccaggaaaccacgactcaagggcccggagtcctgcttcccctgcccaagggggcctgcacaggttggatggcgggcatcccagggcatccgggccataatggggccccaggccgtgatggcagagatggcacccctggtgagaagggtgagaaaggagatccaggtcttattggtcctaagggagacatcggtgaaaccggagtacccggggctgaaggtccccgaggctttccgggaatccaaggcaggaaaggagaacctggagaaggtgcctatgtataccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactgatcaccactaactcagagcctcctccaggccaaacagccccaaagtcaattaaaggctttcagtacggttaggaagttgattattatttagttggaggcctttagatattattcattcatttactcattcatttattcattcattcatcaagtaactttaaaaaaatcatatgctatgttcccagtcctggggagcttcacaaacatgaccagataactgactagaaagaagtagttgacagtgctattttgtgcccactgtctctcctgatgctcatatcaatcctataaggcacagggaacaagcattctcctgtttttacagattgtatcctgaggctgagagagttaagtgaatgtctaaggtcacacagtattaagtgacagtgctagaaatcaaacccagagctgtggactttgttcactagactgtgcccttttatagaggtacatgttctctttggagtgttggtaggtgtctgtttcccacctcacctgagagccattgaatttgccttcctcatgaattaaaacctcccccaagcagagcttcctcagagaaagtggttctatgatgaagtcctgtcttggaaggactactactcaatggcccctgcactactctacttcctcttacctatgtcccttctcatgcctttccctccaacggggaaagccaactccatctctaagtgctgaactcatccctgttcctcaaggccacctggccaggagcttctctgatgtgatatccactttttttttttttgagatggagtctcactctgtcacccaggctggagtacagtgacacgacctcggctcactgcagcctccttctcctgggtccaagcaattattgtgcctcagcctcccgagtagctgagacttcaggtgcattccaccacacatggctaatttttgtatttttagtagaaatggggtttcgtcatgttggccaggctggtctcgaactcctggcctaggtgatccacccgcctcgacctcccaaagtgctgggattacaggcatgagccaccatgcccagtcgatatctcactttttattttgccatggatgagagtcctgggtgtgaggaacacctcccaccaggctagaggcaactgcccaggaaggactgtgcttccgtcacctctaaatcccttgcagatccttgataaatgcctcatgaagaccaatctcttgaatcccatatctacccagaattaactccattccagtctctgcatgtaatcagttttatccacagaaacattttcattttaggaaatccctggttttaagtatcaatccttgttcagctggacaatatgaatcttttccactgaagttagggatgactgtgattttcagaacacgtccagaatttttcatcaagaaggtagcttgagcctgaaatgcaaaacccatggaggaattctgaagccattgtctccttgagtaccaacagggtcagggaagactgggcctcctgaatttattattgttctttaagaattacaggttgaggtagttgatggtggtaaacattctctcaggagacaataactccagtgatgttcttcaaagattttagcaaaaacagagtaaatagcattctctatcaatatataaatttaaaaaactatctttttgcttacagttttaaattctgaacaattctctcttatatgtgtattgctaatcattaaggtattattttttccacatataaagctttgtctttttgttgttgttgttgtttttaagatggagtttccctctgttgccaggctagagtgcagtggcatgatctcggcttactgcaacctttgcctcccaggttcaagcgattcttctgcctcagcctcccgagtagctgggaccacaggtgcctaccaccatgccaggctaatttttgtatttttagtaaagacagggtttcaccatattggccaggctggtctcgaactcctgaccttgtgatctgcccgcctccatttttgttgttattttttgagaaagatagatatgaggtttagagagggatgaagaggtgagagtaagccttgtgttagtcagaactctgtgttgtgaatgtcattcacaacagaaaacccaaaatattatgcaaactactgtaagcaagaaaaataaaggaaaaatggaaacatttattcctttgcataatagaaattaccagagttgttctgtctttagataaggtttgaaccaaagctcaaaacaatcaagacccttttctgtatgtccttctgttctgccttccgcagtgtaggctttaccctcaggtgctacacagtatagttctagggtttccctcccgatatcaaaaagactgtggcctgcccagctctcgtatccccaagccacaccatctggctaaatggacatcatgttttctggtgatgcccaaagaggagagaggaagctctctttcccagatgccccagcaagtgtaaccttgcatctcattgctctggctgagttgtgtgcctgtttctgaccaatcactgagtcaggaggatgaaatattcatattgacttaattgcagcttaagttaggggtatgtagaggtattttccctaaagcaaaattgggacactgttatcagaaataggagagtggatgatagatgcaaaataatacctgtccacaacaaactcttaatgctgtgtttgagctttcatgagtttcccagagagacatagctggaaaattcctattgattttctctaaaatttcaacaagtagctaaagtctggctatgctcacagtctcacatctggttggggtgggctccttacagaacacgctttcacagttaccctaaactctctggggcagggttattcctttgtggaaccagaggcacagagagagtcaactgaggccaaaagaggcctgagagaaactgaggtcaagatttcaggattaatggtcctgtgatgctttgaagtacaattgtggatttgtccaattctctttagttctgtcagcttttgcttcatatattttagcgctctattattagatatatacatgtttagtattatgtcttattggtgcatttactctcttatcattatgtaatgtccttctttatctgtgataattttctgtgttctgaagtctactttgtctaaaaataacatacgcactcaacttccttttctttcttccttcctttctttcttccttcctttctttctctctctctctctttccttccttccttcctccttttctttctctctctctctctctctctttttttgacagactctcgttctgtggccctggctggagttcagtggtgtgatcttggctcactgctacctctaccatgagcaattctcctgcctcagcctcccaagtagctggaactacaggctcatgccactgcgcccagctaatttttgtatttttcgtagagacggggtttcaccacattcgtcaggttggtttcaaactcctgactttgtgatccacccgcctcggcctcccaaagtgctgggattacaggcatgagccatcacacctggtcaactttcttttgattagtgtttttgtggtatatctttttccatcatgttactttaaatatatctatattattgtatttaaaatgtgtttcttacagactgcatgtagttgggtataatttttatccagtctaaaaatatctgtcttttaattggtgtttagacaatttatatttaataaaattgttgaatttaaaaaaaaaaaaaaaa

SEQ ID NO: 4 - DNA, 19 bp, Artificial Sequence
  Description of Artificial Sequence Synthetic primer
gtttgccactcccaagcac

SEQ ID NO: 5 - DNA, 20 bp, Artificial Sequence
  Description of Artificial Sequence Synthetic primer
gtaaagtgcatggtgggtac

SEQ ID NO: 6 - DNA, 25 bp, Artificial Sequence
  Description of Artificial Sequence Synthetic probe
accactcaagccaagtcccaggaac

SEQ ID NO: 7 - DNA, 20 bp, Artificial Sequence
  Description of Artificial Sequence Synthetic primer
cctggcaaatgtgacatctg

SEQ ID NO: 8 - DNA, 20 bp, Artificial Sequence
  Description of Artificial Sequence Synthetic primer
cgtggaagtgaacaaaggca

SEQ ID NO: 9 - DNA, 28 bp, Artificial Sequence
  Description of Artificial Sequence Synthetic probe
cactctcatcagctcttccacatctttg

SEQ ID NO: 10 - DNA, 20 bp, Artificial Sequence
  Description of Artificial Sequence Synthetic primer
cttttatcgctcccagcaga

SEQ ID NO: 11 - DNA, 20 bp, Artificial Sequence
  Description of Artificial Sequence Synthetic primer
ctcgcttcctcatctgcaat

SEQ ID NO: 12 - DNA, 28 bp, Artificial Sequence
  Description of Artificial Sequence Synthetic probe
cgtgattaccaaggttgtcatgaaccca

SEQ ID NO: 13 - DNA, 20 bp, Artificial Sequence
  Description of Artificial Sequence Synthetic primer
tgccaccagtctgaaactca

SEQ ID NO: 14 - DNA, 20 bp, Artificial Sequence
  Description of Artificial Sequence Synthetic primer
cagcaggtctgcaaaccact

SEQ ID NO: 15 - DNA, 25 bp, Artificial Sequence
  Description of Artificial Sequence Synthetic probe
aggcatgcgtgtccctgcacagcca

SEQ ID NO: 16 - DNA, 20 bp, Artificial Sequence
  Description of Artificial Sequence Synthetic primer
agtagtaggagactggacag

SEQ ID NO: 17 - DNA, 21 bp, Artificial Sequence
  Description of Artificial Sequence Synthetic primer
aatgaagggcagaatctcaga

SEQ ID NO: 18 - DNA, 28 bp, Artificial Sequence
  Description of Artificial Sequence Synthetic probe
tccgggatttgctactgcctccatcaag

SEQ ID NO: 19 - DNA, 20 bp, Artificial Sequence
  Description of Artificial Sequence Synthetic primer
aagagctatagactgcctga

SEQ ID NO: 20 - DNA, 20 bp, Artificial Sequence
  Description of Artificial Sequence Synthetic primer
acggatgtcaacgtcacact

SEQ ID NO: 21 - DNA, 24 bp, Artificial Sequence
  Description of Artificial Sequence Synthetic probe
cactattggcaacgagcggttccg